SEQUENCE LISTING (3 sequences)

SEQ ID NO: 1 - DNA, 19 bp, Homo sapiens
gggtacttcattgatgcca

SEQ ID NO: 2 - DNA, 19 bp, Homo sapiens
ggtcagataaaactctcca

SEQ ID NO: 3 - DNA, 19 bp, Homo sapiens
gggcttttcaaaaatgaaa

(19) United States
(12) Patent Application Publication
(10) Pub. No.: US 2008/0019961 A1
(43) Pub. Date: Jan. 24, 2008

(54) Hedgehog signaling pathway antagonist cancer treatment

(75) Inventors: Max S. Wicha, Ann Arbor, MI (US); Gabriela Dontu, Ann Arbor, MI (US); Suling Liu, Ann Arbor, MI (US)

Correspondence Address: Casimir Jones, S.C., 440 Science Drive, Suite 203, Madison, WI, 53711, US

(73) Assignee: Regents of the University of Michigan, Ann Arbor, MI (US)

(21) Appl. No.: 11/708,885

(22) Filed: Feb. 21, 2007

Related U.S. Application Data
(60) Provisional application No. 60/775,302, filed on Feb. 21, 2006.

(51) Int. Cl. A61K 39/395; A61K 31/58; A61P 35/00; C12N 5/00; C12Q 1/02

(52) U.S. Cl. 424/130.1; 435/29; 435/379; 514/176; 514/789

(57) ABSTRACT

The present invention provides methods and compositions for treating tumorigenic cells (e.g., mammary progenitor cancer cells), with hedgehog signaling pathway antagonists (e.g., Cyclopamine or analogs thereof), as well as methods and compositions for screening hedgehog signaling pathway antagonists for their ability serve as anti-neoplastic agents capable of killing tumorigenic cells. The present invention provides methods for identifying tumorigenic cells based on increased expression of a hedgehog signaling pathway component (e.g. PTCH1, Ihh, Gli1, Gli1, Bmi-1, and VEGF), methods of obtaining enriched populations of tumorigenic cells, and methods of causing mammary progenitor cells to proliferate and/or differentiate.

The present application claims priority to U.S. Provisional Application Ser. No. 60/775,302, filed Feb. 21, 2006, which is herein incorporated by reference.

The present invention was made with government support under grant number R01CA101860-02 awarded by the National Institutes of Health. The government has certain rights in the invention.

### FIELD OF THE INVENTION

The present invention relates to methods and compositions for treating tumorigenic cells (e.g., mammary progenitor cancer cells), with hedgehog signaling pathway antagonists (e.g., Cyclopamine or analogs thereof), as well as methods and compositions for screening hedgehog signaling pathway antagonists for their ability serve as anti-neoplastic agents capable of killing tumorigenic cells. The present invention provides methods for identifying tumorigenic cells based on increased expression of a hedgehog signaling pathway component (e.g. PTCH1, Ihh, Gli1, Gli1, Bmi-1, and VEGF), methods of obtaining enriched populations of tumorigenic cells, and methods of causing mammary progenitor cells to proliferate and/or differentiate (e.g. using Sonic Hedgehog, Indian Hedgehog, Gli1, or Gli2).

### BACKGROUND

Cancer is one of the leading causes of death and metastatic cancer is often incurable. Although current therapies can produce tumor regression, they rarely cure common tumors such as metastatic breast cancer (Lippman, M. E., N Engl J Med 342, 1119-20 (2000), herein incorporated by reference). Solid tumors consist of heterogeneous populations of cancer cells. Like acute myeloid leukemia (AML) (Lapidot, T. et al., Nature 17, 645-648 (1994), herein incorporated by reference), it has been demonstrated recently that in most malignant human breast tumors, a small, distinct population of cancer cells are enriched for the ability to form tumors in immunodeficient mice (Al-Hajj et al., Proc Natl Acad Sci USA 100, 3983-8 (2003), herein incorporated by reference). Previously it was shown that in 8 of the 9 tumors studied, the CD44⁺CD24^{-/low}Lineage⁻ population had the ability to form tumors when injected into immunodeficient mice. As few as 200 of these cells, termed “tumorigenic” cells, consistently formed tumors in mice. In contrast, the majority of the cancer cells in a tumor consisted of “non-tumorigenic” cells with alternative phenotypes. These cells failed to form tumors in NOD/SCID mice even when as many as 10⁴ cells were injected (Al-Hajj et al, 2003). In some tumors further enrichment of the tumorigenic cells was possible by isolating the ESA⁺CD44⁺CD24^{-/low}Lineage⁻ population of cancer cells. What is needed therefore, are compositions and methods for treating tumorigenic cells (e.g. tumorigenic breast cancer cells), as well as methods for screening to identify such therapeutic compositions.

### SUMMARY OF THE INVENTION

The present invention provides methods and compositions for treating tumorigenic cells (e.g., mammary progenitor cancer cells), with hedgehog signaling pathway antagonists (e.g., Cyclopamine or analogs thereof), as well as methods and compositions for screening hedgehog signaling pathway antagonists for their ability serve as anti-neoplastic agents capable of killing tumorigenic cells. The present invention provides methods for identifying tumorigenic cells based on increased expression of a hedgehog signaling pathway component (e.g. PTCH1, Ihh, Gli1, Gli1, Bmi-1, and VEGF), methods of obtaining enriched populations of tumorigenic cells, and methods of causing mammary progenitor cells to proliferate and/or differentiate.

In some embodiments, the present invention provides methods of reducing or eliminating tumorigenic cells in a subject, comprising: administering a composition comprising Cyclopamine or Cyclopamine analog to the subject (e.g., under conditions such that at least a portion of said tumorigenic cells are killed, inhibited from proliferating, and/or from causing metastasis). In other embodiments, the present invention provides methods for reducing or eliminating tumorigenic cells in a subject, comprising: administering a hedgehog signaling pathway antagonist to the subject (e.g., under conditions such that at least a portion of said tumorigenic cells are killed, inhibited from proliferating, and/or from causing metastasis). In certain embodiments, the present invention provides methods of treating a subject having a tumorigenic mammary cell, comprising administering a hedgehog signaling pathway antagonist to the subject (e.g., under conditions such that at least a portion of said tumorigenic cells are killed, inhibited from proliferating, or from causing metastasis). In particular embodiments, the administering is under conditions such that the tumorigenic mammary cell is killed. In further embodiments, the present invention provides methods of preventing or reducing metastasis, comprising: administering a hedgehog signaling pathway antagonist to a subject suspected of having metastasis. In particular embodiments, the hedgehog signaling pathway is the Sonic hedgehog, Indian hedgehog, or Desert hedgehog signaling pathway, or the Wnt signaling pathway.

In particular embodiments, the administering is conducted under conditions such that said tumorigenic cells are killed or inhibited from proliferating or causing metastasis. In certain embodiments, the tumorigenic cells are mammary progenitor cells characterized by an increased level of expression of a hedgehog signaling pathway component (e.g., PTCH1, Ihh, Gli1, Gli1, Bmi-1, or VEGF) compared to non-tumorigenic mammary cells from the subject (e.g. from the same tumor biopsy sample). In other embodiments, the tumorigenic cells are mammary progenitor cells. In further embodiments, the hedgehog signaling pathway antagonist comprises an antibody or antibody fragment (e.g. specific for PTCH1, Ihh, Gli1, Gli1, Bmi-1, or VEGF). In some embodiments, the hedgehog signaling pathway antagonist comprises Cylopamine, a Cyclopamine analog, or siRNA molecules, or other antagonists (e.g., antibodies, peptides, small molecules, etc.) configured to disrupt the expression of Bmi-1, PTCH1, Ihh, Gli1, Gli1, Bmi-1, or VEGF.

In particular embodiments, the tumorigenic cells are mammary cells (or other types of tumorigenic cells) characterized by an increased level of expression (e.g. up-regulated) PTCH1, Ihh, Gli1, Gli1, Bmi-1, or VEGF (e.g., as compared to non-tumorigenic mammary cells from the subject). In some embodiments, the methods further comprise determining that the tumorigenic cells have an increased level of PTCH1, Ihh, Gli1, Gli1, Bmi-1, or VEGF (e.g., as compared to non-tumorigenic cells from the subject). In certain embodiments, the tumorigenic or non-tumorigenic cells are mammary cells, cells of epithelia origin, neuronal cells, pancreatic cells, colon cells, etc.).

In certain embodiments, the methods further comprise surgically removing a tumor from the subject prior to the administering step. In other embodiments, the administering further comprises providing a second agent to the subject, where the second agent is anti-neoplastic. In some embodiments, the administering is intravenous and is performed at a distance of no more than 10 inches from the tumorigneic breast cells (e.g. no more than 9, 8, 7, 6, 5, 4, 3, 2 or 1 inches from the targeted tumorigenic breast cells).

In further embodiments, the present invention provides methods for identifying the presence of a progenitor cell (e.g. mammary progenitor) in a sample, comprising: detecting increased expression of PTCH1, Ihh, Gli1, Gli1, Bmi-1, or VEGF in a cell in the sample, and identifying the cell as a progenitor cell. In other embodiments, the present invention provides methods for identifying the presence of a tumorigenic cell in a tumor sample, comprising: detecting increased expression of PTCH1, Ihh, Gli1, Gli1, Bmi-1, or VEGF in a cell in the tumor sample, and identifying the cell as a tumorigenic cell.

In certain embodiments, the tumor sample comprises a breast cancer tumor sample. In other embodiments, the methods further comprise the step of selecting a treatment course of action for a subject based on the presence or absence of the tumorigenic cell in the tumor sample. In further embodiments, the treatment course of action comprises administration of a hedgehog signaling pathway antagonist to the subject. Tumorigenic cells may be detected by any method. For example, detection of markers associated with tumorigenic cancer stem cells, as described, for example, in WO05005601 or co-pending U.S. application Ser. No. 10/864,207, both of which are herein incorporated by reference.

In particular embodiments, the present invention provides methods for screening a compound, comprising: a) exposing a sample comprising a tumorigenic cell (e.g. mammary cell) to a candidate anti-neoplastic compound, wherein the candidate anti-neoplastic compound comprises a hedgehog signaling pathway antagonist; and b) detecting a change in the cell in response to the compound. In some embodiments, the sample comprises a non-adherent mammosphere. In certain embodiments, the hedgehog signaling pathway antagonist comprises an antibody or antibody fragment. In further embodiments, the hedgehog signaling pathway antagonist comprises a Cyclopamine analog. In particular embodiments, the sample comprises human breast tissue. In some embodiments, the detecting comprises detecting cell death of the tumorigenic breast cell. In further embodiments, the methods further comprise identifying the candidate anti-neoplastic agent as capable of killing tumorigenic cells.

In some embodiments, the present invention provides methods of obtaining an enriched population of progenitor cells, comprising a) providing an initial sample comprising progenitor and non-progenitor cells, and b) sorting the initial sample based on the expression level of PTCH 1, Ihh, Gli1, Gli1, Bmi-1, or VEGF in the cells such that an enriched population is generated, wherein the enriched population contains a higher percentage of progenitor cells than present in the initial sample. In certain embodiments, the sorting comprises the use of flow cytometry. In further embodiments, the sorting comprises the use of immuno-magnetic sorting. In other embodiments, the progenitor cells comprise tumorigenic cells and the non-progenitor cells comprise non-tumorigenic cells. In additional embodiments, the progenitor and non-progenitor cells comprise mammary cells.

In other embodiments, the present invention provides methods for expanding a mammary progenitor cell sample, comprising; a) providing a sample (e.g. isolated from an animal) comprising mammary progenitor cells, and b) treating the sample in vitro with a hedgehog signaling pathway agonist under conditions such that the mammary progenitor cells proliferate, differentiate, or proliferate and differentiate. In particular embodiments, the sample comprises a non-adherent mammosphere. In certain embodiments, the agonist is selected from Sonic Hedghog (Shh), Indian Hedgehog (Ihh), Gli1, or Gli2.

In some embodiments, the present invention provides kits comprising; a) a composition comprising a hedgehog signaling pathway antagonist; and b) an insert component comprising instructions for using the composition for treating breast cancer. In preferred embodiments, the hedgehog signaling pathway antagonist comprises Cyclopamine or a Cyclopamine analog.

In certain embodiments, the present invention provides compositions comprising a hedgehog signaling pathway antagonist and a second agent, wherein the second agent is known to reduce or eliminate breast cancer cells when administered to a subject.

### DESCRIPTION OF FIGURES

FIG. 1 shows results from Example 1, and specifically shows mRNA expression of genes in the Hedgehog pathway in mammospheres, differentiated mammary cells, and mammary fibroblasts. Mammary epithelial cells were cultured as mammospheres in suspension or as differentiated mammary cells on collagen substrata, and the mammary fibroblasts from the same patient were cultured on collagen substrata. Total RNA was isolated and mRNA was quantitated by real-time RT-PCR. Data are presented as means ±STDEV. The asterisks indicate statistically significant differences from the differentiated cells (p<0.05). FIG. 1A: mRNA expression of Hedgehog ligands: Sonic Hedgehog (Shh), Indian Hedgehog (Ihh), Desert Hedgehog (Dhh). FIG. 1B: mRNA expression of Hedgehog receptor: PTCH1, PTCH2 and SMO. FIG. 1C: mRNA expression of transcription factors: Gli1 and Gli2. FIG. 1D: Polycomb gene Bmi-1 mRNA expression.

FIG. 2 shows results from Example 1, and specifically shows the effects of activation or inhibition of Hedgehog signaling on mammary stem cell self-renewal. Data are presented as mean ±STDEV. The asterisks show statistically significant differences from the control group (p<0.05).

FIG. 2A: Effects of Hedgehog agonist and antagonist on primary and secondary mammosphere formation. Primary mammospheres were grown in suspension for 7-10 days in the presence or absence of 3 μg/ml of Sonic Hedgehog (Shh), 300 nM of Cyclopamine (CP) or 5 μM of γ-secretase inhibitor (GSI), which is Z-Leu-Leu-Nle-CHO; Calbiochem, San Diego, Calif. Single cells dissociated from each group were grown as secondary mammospheres in suspension for 7-10 days without treatment. The # of mammospheres represents the total mammospheres formed from 10,000 single cells; the # of cells represents the total single cells dissociated from one mammosphere.

FIG. 2B: Effects of Gli1 and Gli2 overexpression on mammary stem cell self-renewal. Secondary mammospheres were infected with SIN-IP-EGFP virus, SIN-GLI1-EGFP virus, SIN-GLI2-EGFP virus or none as the control.

FIG. 3 shows results from Example 1, and specifically shows the effects Hh signaling on branching morphogenesis. Data are presented as mean ±STDEV. The asterisks show statistically significant differences from the control group (p<0.05).

FIG. 3A: Effects of Hh agonist and antagonist on mammosphere branching morphogenesis in 3-D matrigel culture. Primary mammospheres were grown in the presence or absence of 3 μg/ml of Sonic Hedgehog (Shh), 300 nM of Cyclopamine (CP) for 7-10 days. Then, 30 mammospheres per well of 24-well plates were used in 3-D matrigel culture and each group of mammospheres was performed in quadruplicates. FIG. 3B: Effects of Gli1 and Gli2 on mammosphere branching morphogenesis in 3-D matrigel culture. Single cells from primary mammospheres were infected with SIN-IP-EGFP, SIN-GLI1-EGFP, or SIN-GLI2-EGFP virus, or un-infected (Non) as the control, and cultured in suspension for 7-10 days. Then, 3-D matrigel culture was performed as described in A.

FIG. 4 shows results from Example 1, and specifically shows the effects of Hh signaling activation on the mammary outgrowth of engrafted human mammospheres in NOD/SCID mice and angiogenesis. FIGS. 4A and 4B: Whole-mount analysis for SIN-IP-EGFP virus (A) or SIN-GLI2-EGFP virus (B) infected mammosphere xenograft outgrowth. FIGS. 4C, 4D, 4E, and 4F: H&E staining for SIN-IP-EGFP virus (C and E) or SIN-GLI2-EGFP virus (D and F) infected mammosphere xenograft outgrowth. Arrow: hyperplastic structures. FIGS. 4E and 4F: Blood vessel formation in SIN-IP-EGFP virus (E) or SIN-GLI2-EGFP virus (F) infected mammosphere xenograft outgrowth. Arrow: blood vessels. Bar: 100 μm. FIG. 4G: Effects of Shh on VEGF production. Primary mammospheres were grown in the presence or absence of 3 μg/ml of Sonic Hedgehog (Shh) for 7-10 days. Total RNA was isolated and mRNA was quantitated by real-time RT-PCR. Data are presented as mean ±STDEV. The asterisks show statistically significant differences from the control group (p<0.05). FIG. 4H: Effects of Gli-overexpression on VEGF production. Single cells from primary mammospheres were infected with SIN-IP-EGFP (Shh) or inhibited with 300 nM Cyclopamine (CP) or 5 μM γ-secretase inhibotor (GSI), or activated by Gli overexpression. Notch pathway was activated with 10 μM Delta/Serrate/LAG-2 (DSL) or inhibited with 5 μM GSI or 300 nM Cyclopamine; Data is presented as mean ±STDEV. The asterisks show statistically significant differences from the control group (p<0.05). FIG. 5A: Effects of Hedgehog signaling on PTCH1, Gli1, Gli2 and HES1 as determined by real-time RT-PCR. FIG. 5B: Effects of Notch signaling on HES1, PTCH1, Gli1 and Gli2 mRNA expression as determined by real-time RT-PCR. FIG. 5C: Effects of Hedgehog signaling and Notch signaling on Bmi-1 mRNA expression.

FIG. 6 shows results from Example 1, and specifically shows the effects of activation or inhibition of Hedgehog or Notch signaling on self-renewal of mammary stem cells. Data are presented as mean ±STDEV. The asterisks show statistically significant differences from the control group (p<0.05). FIG. 6A: Effect of Hedgehog agonist and antagonist treatment on primary and secondary mammosphere formation. Primary mammospheres were grown in the presence or absence of 3 μg/ml of Sonic Hedgehog (Shh), 300 nM of Cyclopamine (CP) or 5 μM of γ-secretase inhibotor (GSI). The # of mammospheres was the total mammospheres formed from 10,000 single cells; the # of cells was the total single cells dissociated from one mammosphere. FIG. 6B: Effect of Notch agonist and antagonist treatment on primary and secondary mammosphere formation. Primary mammospheres were grown in the presence or absence of 10 μM of Delta/Serrate/LAG-2 (DSL), 5 μM of γ-secretase inhibotor (GSI) or 300 nM of Cyclopamine (CP). The # of mammospheres was the total mammospheres formed from 10000 single cells; the # of cells was the total single cells dissociated from one mammosphere.

FIG. 7 shows results from Example 1, and specifically shows knock-down of Bmi-1 expression by Bmi-1 siRNA lentiviruses in mammosphere culture system. Primary mammospheres were infected with the control virus (HIV-GFP-VSVG) or siRNA lentiviruses (HIV-siRNA1-VSVG, HIV-siRNA2-VSVG, HIV-siRNA3-VSVG), or un-infected (Non) as the control, and cultured in suspension for 7 days. Total RNA and total protein were isolated, and mRNA was quantitated by real-time RT-PCR and protein was quantitated by western blotting. FIG. 7A: Human Bmi-1 mRNA expression analyzed by real-time RT-PCR. Data is presented as mean ±STDEV. The asterisks show statistically significant differences from the control group (p<0.05). FIG. 7B: Human Bmi-1 protein expression analyzed by western blotting.

FIG. 8 shows results from Example 1, and specifically shows the effects of Bmi-1 on the regulation of mammary stem cell self-renewal by Hh and Notch signaling. Data is presented as mean ±STDEV. The asterisks or & show statistically significant differences from the control group (p<0.05) or untreated group (&<0.05), respectively. FIG. 8A: Primary mammospheres were infected with the control virus (HIV-GFP-VSVG) or siRNA lentiviruses (HIV-siRNA1-VSVG, HIV-siRNA2-VSVG, HIV-siRNA3-VSVG), or uninfected (Non) as the control, and cultured in suspension in the absence (untreated) or presence of 3 μg/ml Sonic hedgehog (Shh) or 10 μM of Delta/Serrate/LAG-2 (DSL) for 7-10 days. The total mammospheres formed from 10,000 single cells and the total single cells dissociated from one mammosphere were counted and graphed. FIG. 8B: The single cells dissociated from each group in A were grown as secondary mammospheres in suspension for 7-10 days without treatment. The # of secondary mammospheres was the total mammospheres formed from 10,000 single cells; the # of cells was the total single cells dissociated from one secondary mammosphere.

FIG. 9 shows results from Example 1, and specifically shows Hh signaling in breast tumorigenesis and angiogenesis. FIG. 9A: Tumor cells were isolated from the mouse xenografts, both CD44+CD24−/lowlinpopulation and CD44−/lowCD24+lin+population were sorted by flow cytometry. Total RNA was isolated and mRNA for Hh component gene and Bmi-1 was quantitated by realtime RT-PCR. Data is presented as mean ±STDEV. The asterisks show statistically significant differences from the control group (p<0.05). FIG. 9B: Phenotypic diversity in tumors arising from total tumor cells. FIG. 9C: Phenotypic diversity in tumors arising from PTCH1+Ihh+tumor cells. FIG. 9D: Sorted PTCH1+Ihh+tumor cells and PTCH1−Ihh−tumor cells were injected into the fat pads of NOD-SCID mice. Identical number of both populations was injected into the different side of mammary fat pads in the same mouse. The tumor growth was observed every week and the tumors were removed at 8th week after injection. FIG. 9E: Tumor cells were isolated from the mouse xenografts, both PTCH1+Ihh+tumor cells and PTCH1−Ihh−tumor cells were sorted by flow cytometry. Total RNA was isolated and mRNA for Bmi-1 was quantitated by real-time RT-PCR. Data is presented as mean ±STDEV. The asterisks show statistically significant differences from the control group (p<0.05). FIG. 9F: Tumor cells were isolated from the mouse xenografts, both PTCH1+Ihh+tumor cells and PTCH1−Ihh−tumor cells were sorted by flow cytometry. Total RNA was isolated and mRNA for VEGF was quantitated by real-time RT-PCR. Data is presented as mean ±STDEV. The asterisks show statistically significant differences from the control group (p<0.05).

### DEFINITIONS

To facilitate an understanding of the present invention, a number of terms and phrases are defined below:

As used herein, the phrase “hedgehog signaling pathway antagonist” includes any compound or agent that prevents signal transduction in the hedgehog signaling pathway, and specifically includes any compound that inhibits hedgehog from binding with its receptor. Examples of such compounds include, but are not limited to, Cyclopamine, Cyclopamine analogs, and siRNA molecules configured to disrupt the expression of Bmi-1 (for BMI-1 siRNA methods and materials, see Zencak et al., The Journal of Neuroscience; Jun. 15, 2005, 25(24):5774-5783, and Bracken et al., The EMBO Journal, Vol. 22, No. 20 pp. 5323-5335, 2003, both of which are herein incorporated by reference).

As used herein, the terms “anticancer agent,” “conventional anticancer agent,” or “cancer therapeutic drug” refer to any therapeutic agents (e.g., chemotherapeutic compounds and/or molecular therapeutic compounds), radiation therapies, or surgical interventions, used in the treatment of cancer (e.g., in mammals).

As used herein, the terms “drug” and “chemotherapeutic agent” refer to pharmacologically active molecules that are used to diagnose, treat, or prevent diseases or pathological conditions in a physiological system (e.g., a subject, or in vivo, in vitro, or ex vivo cells, tissues, and organs). Drugs act by altering the physiology of a living organism, tissue, cell, or in vitro system to which the drug has been administered. It is intended that the terms “drug” and “chemotherapeutic agent” encompass anti-hyperproliferative and antineoplastic compounds as well as other biologically therapeutic compounds.

As used herein the term “prodrug” refers to a pharmacologically inactive derivative of a parent “drug” molecule that requires biotransformation (e.g., either spontaneous or enzymatic) within the target physiological system to release, or to convert (e.g., enzymatically, mechanically, electromagnetically, etc.) the “prodrug” into the active “drug.” “Prodrugs” are designed to overcome problems associated with stability, toxicity, lack of specificity, or limited bioavailability. Exemplary “prodrugs” comprise an active “drug” molecule itself and a chemical masking group (e.g., a group that reversibly suppresses the activity of the “drug”). Some preferred “prodrugs” are variations or derivatives of compounds that have groups cleavable under metabolic conditions. Exemplary “prodrugs” become pharmaceutically active in vivo or in vitro when they undergo solvolysis under physiological conditions or undergo enzymatic degradation or other biochemical transformation (e.g., phosphorylation, hydrogenation, dehydrogenation, glycosylation, etc.). Prodrugs often offer advantages of solubility, tissue compatibility, or delayed release in the mammalian organism. (See e.g., Bundgard, Design of Prodrugs, pp. 7-9, 21-24, Elsevier, Amsterdam (1985); and Silverman, The Organic Chemistry of Drug Design and Drug Action, pp. 352-401, Academic Press, San Diego, Calif. (1992)). Common “prodrugs” include acid derivatives such as esters prepared by reaction of parent acids with a suitable alcohol (e.g., a lower alkanol), amides prepared by reaction of the parent acid compound with an amine (e.g., as described above), or basic groups reacted to form an acylated base derivative (e.g., a lower alkylamide).

An “effective amount” is an amount sufficient to effect beneficial or desired results. An effective amount can be administered in one or more administrations.

As used herein, the term “administration” refers to the act of giving a drug, prodrug, antibody, or other agent, or therapeutic treatment to a physiological system (e.g., a subject or in vivo, in vitro, or ex vivo cells, tissues, and organs). Exemplary routes of administration to the human body can be through the eyes (opthalmic), mouth (oral), skin (transdermal), nose (nasal),.lungs (inhalant), oral mucosa (buccal), ear, by injection (e.g., intravenously, subcutaneously, intratumorally, intraperitoneally, etc.) and the like.

“Coadministration” refers to administration of more than one chemical agent or therapeutic treatment (e.g., radiation therapy) to a physiological system (e.g., a subject or in vivo, in vitro, or ex vivo cells, tissues, and organs). “Coadministration” of the respective chemical agents (e.g. hedgehog signaling pathway antagonist) and therapeutic treatments (e.g., radiation therapy) may be concurrent, or in any temporal order or physical combination.

As used herein, the term “bioavailability” refers to any measure of the ability of an agent to be absorbed into a biological target fluid (e.g., blood, cytoplasm, CNS fluid, and the like), tissue, organelle or intercellular space after administration to a physiological system (e.g., a subject or in vivo, in vitro, or ex vivo cells, tissues, and organs).

As used herein, the term “biodistribution” refers to the location of an agent in organelles, cells (e.g., in vivo or in vitro), tissues, organs, or organisms, after administration to a physiological system.

A “hyperproliferative disease,” as used herein refers to any condition in which a localized population of proliferating cells in an animal is not governed by the usual limitations of normal growth. Examples of hyperproliferative disorders include tumors, neoplasms, lymphomas and the like. A neoplasm is said to be benign if it does not undergo invasion or metastasis and malignant if it does either of these. A “metastatic” cell or tissue means that the cell can invade and destroy neighboring body structures. Hyperplasia is a form of cell proliferation involving an increase in cell number in a tissue or organ without significant alteration in structure or function. Metaplasia is a form of controlled cell growth in which one type of fully differentiated cell substitutes for another type of differentiated cell. Metaplasia can occur in epithelial or connective tissue cells. A typical metaplasia involves a somewhat disorderly metaplastic epithelium.

As used herein, the term “neoplastic disease” refers to any abnormal growth of cells or tissues being either benign (non-cancerous) or malignant (cancerous).

As used herein, the term “anti-neoplastic agent” refers to any compound that retards the proliferation, growth, or spread of a targeted (e.g., malignant) neoplasm.

As used herein, the term “regression” refers to the return of a diseased subject, cell, tissue, or organ to a non-pathological, or less pathological state as compared to basal nonpathogenic exemplary subject, cell, tissue, or organ. For example, regression of a tumor includes a reduction of tumor mass as well as complete disappearance of a tumor or tumors.

As used herein, the terms “prevent,” “preventing,” and “prevention,” in the context of regulation of hyper-proliferation, refer to a decrease in the occurrence of hyperproliferative or neoplastic cells in a subject. The prevention may be complete, e.g., the total absence of hyperproliferative or neoplastic cells in a subject. The prevention may also be partial, such that the occurrence of hyperproliferative or neoplastic cells in a subject is less than that which would have occurred without an intervention.

As used herein the term, “in vitro” refers to an artificial environment and to processes or reactions that occur within an artificial environment. In vitro environments can consist of, but are not limited to, test tubes and cell cultures. The term “in vivo” refers to the natural environment (e.g., an animal or a cell) and to processes or reactions that occur within a natural environment.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro, including oocytes and embryos.

As used herein, the term “subject” refers to organisms to be treated by the methods of the present invention. Such organisms include, but are not limited to, humans and veterinary animals (dogs, cats, horses, pigs, cattle, sheep, goats, and the like). In the context of the invention, the term “subject” generally refers to an individual who will receive or who has received treatment.

The term “diagnosed,” as used herein, refers to the recognition of a disease by its signs and symptoms or genetic analysis, pathological analysis, histological analysis, and the like.

As used herein, the term “competes for binding” is used in reference to a first molecule with an activity that binds to the same target as does a second molecule. The efficiency (e.g., kinetics or thermodynamics) of binding by the first molecule may be the same as, or greater than, or less than, the efficiency of the target binding by the second molecule. For example, the equilibrium binding constant (Kd) for binding to the target may be different for the two molecules.

As used herein, the term “antisense” is used in reference to nucleic acid sequences (e.g., RNA, phosphorothioate DNA) that are complementary to a specific RNA sequence (e.g., mRNA). Included within this definition are natural or synthetic antisense RNA molecules, including molecules that regulate gene expression, such as small interfering RNAs or micro RNAs.

The term “test compound” or “candidate compound” refers to any chemical entity, pharmaceutical, drug, and the like, that can be used to treat or prevent a disease, illness, sickness, or disorder of bodily function, or otherwise alter the physiological or cellular status of a sample. Test compounds comprise both known and potential therapeutic compounds. A test compound can be determined to be therapeutic by using the screening methods of the present invention. A “known therapeutic compound” refers to a therapeutic compound that has been shown (e.g., through animal trials or prior experience with administration to humans) to be effective in such treatment or prevention. In preferred embodiments, “test compounds” are anticancer agents. In particularly preferred embodiments, “test compounds” are anticancer agents that induce apoptosis in cells.

As used herein, the term “antigen binding protein” refers to proteins which bind to a specific antigen. “Antigen binding proteins” include, but are not limited to, immunoglobulins, including polyclonal, monoclonal, chimeric, single chain, and humanized antibodies, Fab fragments, F(ab′)2 fragments, and Fab expression libraries. Various procedures known in the art are used for the production of polyclonal antibodies. For the production of antibodies, various host animals can be immunized by injection with the peptide corresponding to the desired epitope including, but not limited to, rabbits, mice, rats, sheep, goats, etc. In a preferred embodiment, the peptide is conjugated to an immunogenic carrier (e.g., diphtheria toxoid, bovine serum albumin (BSA), or keyhole limpet hemocyanin (KLH)). Various adjuvants are used to increase the immunological response, depending on the host species, including, but not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (Bacille Calmette-Guerin) and Corynebacterium parvum.

For preparation of monoclonal antibodies, any technique that provides for the production of antibody molecules by continuous cell lines in culture may be used (See e.g., Harlow and Lane, Antibodies: A Laboratory Manual, Cold Spring Harbor Laboratory Press, Cold Spring Harbor, N.Y.). These include, but are not limited to, the hybridoma technique originally developed by Kohler and Milstein (Kohler and Milstein, Nature, 256:495-497 (1975)), as well as the trioma technique, the human B-cell hybridoma technique (See e.g., Kozbor et al., Immunol. Today, 4:72 (1983)), and the EBV-hybridoma technique to produce human monoclonal antibodies (Cole et al., in Monoclonal Antibodies and Cancer Therapy, Alan R. Liss, Inc., pp. 77-96 (1985)).

According to the invention, techniques described for the production of single chain antibodies (U.S. Pat. No. 4,946,778; herein incorporated by reference) can be adapted to produce specific single chain antibodies as desired. An additional embodiment of the invention utilizes the techniques known in the art for the construction of Fab expression libraries (Huse et al., Science, 246:1275-1281 (1989)) to allow rapid and easy identification of monoclonal Fab fragments with the desired specificity.

Antibody fragments that contain the idiotype (antigen binding region) of the antibody molecule can be generated by known techniques. For example, such fragments include, but are not limited to: the F(ab′)2 fragment that can be produced by pepsin digestion of an antibody molecule; the Fab′ fragments that can be generated by reducing the disulfide bridges of an F(ab′)2 fragment, and the Fab fragments that can be generated by treating an antibody molecule with papain and a reducing agent.

Genes encoding antigen-binding proteins can be isolated by methods known in the art. In the production of antibodies, screening for the desired antibody can be accomplished by techniques known in the art (e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), Western Blots, precipitation reactions, agglutination assays (e.g., gel agglutination assays, hemagglutination assays, etc.), complement fixation assays, immunofluorescence assays, protein A assays, and immunoelectrophoresis assays, etc.) etc.

As used herein, the term “modulate” refers to the activity of a compound to affect (e.g., to promote or retard) an aspect of the cellular function including, but not limited to, cell growth, proliferation, invasion, angiogenesis, apoptosis, and the like.

### DETAILED DESCRIPTION OF THE INVENTION

The present invention provides methods and compositions for treating tumorigenic cells (e.g., mammary progenitor cancer cells), with hedgehog signaling pathway antagonists (e.g., Cyclopamine or analogs thereof), as well as methods and compositions for screening hedgehog signaling pathway antagonists for their ability serve as anti-neoplastic agents capable of killing tumorigenic cells. The present invention provides methods for identifying tumorigenic cells based on increased expression of a hedgehog signaling pathway component (e.g. PTCH1, Ihh, Gli1, Gli1, Bmi-1, and VEGF), methods of obtaining enriched populations of tumorigenic cells, and methods of causing mammary progenitor cells to proliferate and/or differentiate (e.g. using Sonic Hedgehog, Indian Hedgehog, Gli1, or Gli2).

As described in the Example below, it has been demonstrated that components of Hh signaling, including PTCH1, Gli1, and Gli2 are highly expressed in mammary stem and progenitor cells in mammospheres compared to cells induced to differentiate by attachment to a collagen substratum. Furthermore, it has been determined that activation of this pathway with Hh ligands promotes the selfrenewal of mammary stem cells, as evidenced by an increase in the number of mammosphere initiating multipotent cells. This effect was blocked by Cyclopamine, a specific inhibitor of this pathway. Hh activation also increases the proliferation of mammary progenitor cells as reflected by an increase in mammosphere size.

As described in the Example below, it has been determined that the addition of Hh ligands increase the expression of the transcription factors Gli1 and Gli2 which was inhibited by Cyclopamine. Forced overexpression of Gli1 or Gli2 in mammosphere initiating cells by retroviral transduction, recapulated the effects of Hh ligands. These effects were unaffected by Cyclopamine indicating that Gli1 and Gli2 act downstream of smoothened. Overexpression of Gli1 and Gli2 in mammospheres also increase mammosphere size and promotes branching morphogenesis of these cells in three dimensional matrix based culture systems. This indicates that, in addition to effects on stem cell self-renewal, the Hh pathway also plays a role in progenitor cell proliferation and morphogenetic development. Furthermore, these studies indicate that the effects of Hh activation on primitive mammary cells are mediated by the transcription factors Gli1 and Gli2.

In order to determine if there are interactions between Hh and Notch signaling in mammary stem cells, as described in the Example below, agonist and antagonist of the Notch and Hedgehog pathways were utilized to examine their effects on the alternative pathway. It was demonstrated that activation of the Notch pathway by the Notch ligand DSL induced Hh components PTCH1, Gli1, and Gli2 which could be inhibited by the Notch inhibitor GSI but not by Cyclopamine. Alternatively, activation of Hh signaling with sonic Hh (Shh) increased expression of the Notch pathway target HES 1 which was inhibited with the Hh pathway inhibitor Cyclopamine, but not by GSI. Together, these studies indicate that the Hh and Notch pathways are interconnected with bi-directional signaling occurring between these pathways.

It has been determined that Bmi-1 is expressed at increased levels in undifferentiated compared to differentiated mammary cells. Activation of either Hh or Notch signaling increases Bmi-1 expression. In contrast down-regulation of Bmi-1 utilizing siRNA abrogates the effects of Hh or Notch signaling on mamnmosphere formation. This indicates that the effects of Hh and Notch signaling on mammary stem cell self-renewal are mediated by Bmi-1.

It has been determined that that overexpression of the Hh target Gli2 in mammospheres produces ductal hyperplasias when these cells are implanted into the humanized cleared fat pads of NOD-SCID mice. These findings are consistent with a stem cell model of carcinogenesis in which early events involve deregulation of Hh signaling resulting in clonal expansion of stem or progenitor cells. These cells in turn may undergo further mutations to acquire a fully malignant phenotype. It was also determined that activation of Hh signaling results in increased expression of VEGF.

It has been demonstrated that tumorigenic cells (“tumor stem cells”) display activation of Hh signaling components as well as increased expression of Bmi-1. Cells simultaneously expressing the Hh ligand Ihh as well as its receptor PTCH1 were significantly more tumorigenic than cells isolated from the same tumor which did not express these proteins. PTCH1+Ihh+tumor cells expressed 8-fold higher levels of Bmi-1 than did PTCH1−Ihh−tumor cells. Consistent with a “tumor stem cell model” when PTCH1+Ihh+tumor cells were injected into NOD-SCID mice, they produced tumors which were composed of heterogeneous cell populations which recapitulated the phenotypic heterogeneity found in the initial tumor. Thus, these cells exhibited properties of “tumor stem cells” as evidenced by their ability to undergo self-renewal through multiple passages in NOD-SCID mice as well as differentiation as evidenced by their ability to generate phenotypic heterogeneity.

I. Tumorigenic Cancer Cells

Solid tumors consist of heterogeneous populations of cancer cells that differ in their ability to form new tumors. Cancer cells that have the ability to form tumors (i.e., tumorigenic cancer cells) and cancer cells that lack this capacity (i.e., non-tumorigenic cancer cells) can be distinguished based on phenotype (Al-Hajj, et al., Proc Natl Acad Sci USA 100, 3983-8 (2003); Pat. Pub. 20020119565; Pat. Pub. 20040037815; Pat. Pub. 20050232927; WO05/005601; Pat. Pub. 20050089518; U.S. application Ser. No. 10/864,207; Al-Hajj et al., Oncogene, 2004, 23:7274; and Clarke et al., Ann Ny Acad. Sci., 1044:90, 2005, all of which are herein incorporated by reference in their entireties for all purposes).

The present invention relates to compositions and methods for characterizing, regulating, diagnosing, and treating cancer. For example, the present invention provides compositions and methods for inhibiting tumorigenesis of certain classes of cancer cells, including breast cancer cells and preventing metastasis (e.g., using hedgehog signaling pathway antagonists). The present invention also provides systems and methods for identifying compounds that regulate tumorigenesis. For example, the present invention provides methods for identifying tumorigenic cells and diagnosing diseases (e.g., hyperproliferative diseases) or biological events (e.g., tumor metastasis) associated with the presence of tumorigenic cells. In particular, the present invention identifies classes of cells within cancers that are tumorigenic and provides detectable characteristics of such cells (e.g. up regulated expression of PTCH1, Ihh, Gli1, Gli1, Bmi-1, and VEGF), such that their presence can be determined, for example, in choosing whether to submit a subject to a medical intervention, selecting an appropriate treatment course of action, monitoring the success or progress of a therapeutic course of action (e.g., in a drug trial or in selecting individualized, ongoing therapy), or screening for new therapeutic compounds or therapeutic targets.

In some embodiments, the expression of a hedgehog signaling pathway component is used to identify tumorigenic cells. Regulators of a hedgehog signaling pathway components also find use in research, drug screening, and therapeutic methods. For example, hedgehog signaling pathway antagonists and antagonists of the hedgehog signaling pathways find use in preventing or reducing cell proliferation, hyperproliferative disease development or progression, and cancer metastasis. In some embodiments, antagonists are utilized following removal of a solid tumor mass to help reduce proliferation and metastasis of remaining hyperproliferative cells.

The present invention is not limited to any particular type of tumorigenic cell type, nor is the present invention limited by the nature of the compounds or factors used to regulate tumorigenesis. Thus, while the present invention is illustrated below using breast cancer cells, skilled artisans will appreciate that the present invention is not limited to these illustrative examples. For example, it is contemplated that are variety of neoplastic conditions benefit from the teachings of the present invention, including, but not limited to, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms′ tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

The observation that tumors contain a small population of tumorigenic cells with a common cell surface phenotype (e.g. up-regulated expression of a hedgehog signaling pathway component, such as PTCH1, Ihh, Gli1, Gli1, Bmi-1, and VEGF) has important implications for understanding solid tumor biology and also for the development of effective cancer therapies. The inability of current cancer treatments to cure metastatic disease may be due to ineffective killing of tumorigenic cells. If the tumorigenic cells are spared by an agent, then tumors may regress but the remaining tumorigenic cells will drive tumor recurrence. By focusing on the tumorigenic population, one can identify and affect critical proteins involved in essential biological functions in the tumorigenic population of cancer cells, such as self-renewal and survival.

II. Hedgehog Signaling Pathway Agonists and Antagonists

The methods and compositions of the present invention contemplate the use of compounds that can serve as hedgehog signaling pathway agonists, including Shh, Ihh, Gli1, and Gli2, as well as variants of these agonists, and other compounds that have similar activity (or superior activity) to these agonists. In certain embodiments, the hedgehog signaling pathway agonist is used to cause the proliferation, differentiation, or proliferation and differentiation of progenitor cells, such as mammary progenitor cells.

In certain embodiments, the methods and compositions of the present invention employed a variant of Shh, Ihh, Gli1, and Gli2. Examples of such variants include, but are not limited to, truncated versions of the full length Shh, Ihh, Gli1, and Gli2, and mutated versions with substitutions and/or deletions. Additional hedgehog signaling agonists may be found in the following references: Paladini et al., J Invest Dermatol. 2005 October;125(4):638-46; Frank-Kamenetsky et al., J Biol. 2002 Nov. 6;1(2):10; U.S. Pat. Pub. 20050070578; U.S. Pat. Pub. 20030139457; U.S. Pat. Pub. 20050112125; and U.S. Pat. Pub. 20050054568; all of which are herein. incorporated by reference.

The methods and compositions of the present invention also contemplate the use of hedgehog signaling pathway antagonists such as Cyclopamine, as well as antagonists with similar (or increased) anti-tumorigenic activity as Cyclopamine. Exemplary antagonists include, but are not limited to, the Cyclopamine analogs cyclopamine-4-ene-3-one, and Sigma Chemical Product Code J 4145 (see Williams et al., PNAS USA 100, 4616-4621, 2003, herein incorporated by reference). Additional analogs include Cur61414, 5E1 mab, HIP, Frzb, Cerberus, WIF-1, Xnr-3, Gremlin, Follistatin or a derivative, fragment, variant, mimetic, homologue or analogue thereof, Ptc, Cos2, PKA, and an agent of the cAMP signal transduction pathway. References that describe additional antagonists include: U.S. Pat. Pub. 20050112125; Chen et al., Proc. Nat. Acad. Sci. 2002, 99:22, 14071-14076; Taipale et al., Nature 2002, 418, 892-897; Taipale et al., Nature 2000, 406, 1005-1009; U.S. Pat. Pub. 20050222087; U.S. Pat. Pub. 20050085519; U.S. Pat. Pub. 20040127474; U.S. Pat. Pub. 20040110663; U.S. Pat. Pub. 20040038876; and U.S. Pat. Pub. 20030166543; all of which are herein incorporated by reference in their entirities, and particularly for the hedgehog signaling agents taught therein.

III. Non-Adherent Mammospheres and Antagonist Screening

In certain embodiments, the present invention employs non-adherent mammospheres for various screening procedures, including; methods for screening hedgehog signaling pathway antagonists (e.g. to determine if they have similar activity to Cyclopamine), and screening hedgehog signaling pathway agonists to do determine if they have similar activity as Sonic Hedgehog, Indian Hedgehog, Gli1 or Gli2 (e.g. to determine if they are able to cause proliferation and/or differentiation of progenitor cells, such as mammary progenitor cells).

Non-adherent mammospheres are an in vitro culture system that allows for the propagation of primary human mammary epithelial stem and progenitor cells in an undifferentiated state, based on their ability to proliferate in suspension as spherical structures. Non-adherent mammospheres have previously been described in Dontu et al Genes Dev. 2003 May 15;17(10):1253-70, and Dontu et al., Breast Cancer Res. 2004;6(6):R605-15, both of which are herein incorporated by reference. These references are incorporated by reference in their entireties and specifically for teaching the construction and use of non-adherent mammospheres. As described in Dontu et al., mammospheres have been characterized as being composed of stem and progenitor cells capable of self-renewal and multi-lineage differentiation. Dontu et al. also describes that mammospheres contain cells capable of clonally generating complex functional ductal-alveolar structures in reconstituted 3-D culture systems in Matrigel.

IV. Therapeutic Compositions and Administration

A pharmaceutical composition containing a regulator of tumorigenesis according the present invention can be administered by any effective method. For example, a hedgehog signaling pathway antagonist, or other therapeutic agent that acts as an antagonist of proteins in the hedgehog signal transduction/response pathway can be administered by any effective method. For example, a physiologically appropriate solution containing an effective concentration of a hedgehog signaling pathway antagonist can be administered topically, intraocularly, parenterally, orally, intranasally, intravenously, intramuscularly, subcutaneously or by any other effective means. In particular, the hedgehog signaling pathway antagonist agent may be directly injected into a target cancer or tumor tissue by a needle in amounts effective to treat the tumor cells of the target tissue. Alternatively, a cancer or tumor present in a body cavity such as in the eye, gastrointestinal tract, genitourinary tract (e.g., the urinary bladder), pulmonary and bronchial system and the like can receive a physiologically appropriate composition (e.g., a solution such as a saline or phosphate buffer, a suspension, or an emulsion, which is sterile) containing an effective concentration of a hedgehog signaling pathway antagonist via direct injection with a needle or via a catheter or other delivery tube placed into the cancer or tumor afflicted hollow organ. Any effective imaging device such as X-ray, sonogram, or fiber-optic visualization system may be used to locate the target tissue and guide the needle or catheter tube. In another alternative, a physiologically appropriate solution containing an effective concentration of a hedgehog signaling pathway antagonist can be administered systemically into the blood circulation to treat a cancer or tumor that cannot be directly reached or anatomically isolated.

Such manipulations have in common the goal of placing the hedgehog signaling pathway antagonist in sufficient contact with the target tumor to permit the hedgehog signaling pathway antagonist to contact, transduce or transfect the tumor cells (depending on the nature of the agent). In one embodiment, solid tumors present in the epithelial linings of hollow organs may be treated by infusing the suspension into a hollow fluid filled organ, or by spraying or misting into a hollow air filled organ. Thus, the tumor cells (such as a solid tumor stem cells) may be present in or among the epithelial tissue in the lining of pulmonary bronchial tree, the lining of the gastrointestinal tract, the lining of the female reproductive tract, genitourinary tract, bladder, the gall bladder and any other organ tissue accessible to contact with the hedgehog signaling pathway antagonist. In another embodiment, the solid tumor may be located in or on the lining of the central nervous system, such as, for example, the spinal cord, spinal roots or brain, so that the hedgehog signaling pathway antagonist infused in the cerebrospinal fluid contacts and transduces the cells of the solid tumor in that space. One skilled in the art of oncology can appreciate that the hedgehog signaling pathway antagonist can be administered to the solid tumor by direct injection into the tumor so that the hedgehog signaling pathway antagonist contacts and affects the tumor cells inside the tumor.

The tumorigenic cells identified by the present invention can also be used to raise anti-cancer cell antibodies. In one embodiment, the method involves obtaining an enriched population of tumorigenic cells or isolated tumorigenic cells; treating the population to prevent cell replication (for example, by irradiation); and administering the treated cell to a human or animal subject in an amount effective for inducing an immune response to solid tumor stem cells. For guidance as to an effective dose of cells to be injected or orally administered; see, U.S. Pat. Nos. 6,218,166, 6,207,147, and 6,156,305, incorporated herein by reference. In another embodiment, the method involves obtaining an enriched population of solid tumor stem cells or isolated solid tumor stem cells; mixing the tumor stem cells in an in vitro culture with immune effector cells (according to immunological methods known in the art) from a human subject or host animal in which the antibody is to be raised; removing the immune effector cells from the culture; and transplanting the immune effector cells into a host animal in a dose that is effective to stimulate an immune response in the animal.

In some embodiments, the therapeutic agent is an antibody. Monoclonal antibodies to may be prepared using any technique which provides for the production of antibody molecules by continuous cell lines in culture. These include, but are not limited to, the hybridoma technique, the human B-cell hybridoma technique, and the EBV-hybridoma technique (see, e.g., Kozbor, D. et al., J. Immunol. Methods 81:31-42 (1985); Cote R J et al. Proc. Natl. Acad. Sci. 80:2026-2030 (1983); and Cole S P et al. Mol. Cell Biol. 62:109-120 (1984)).

In addition, techniques developed for the production of “chimeric antibodies,” such as the splicing of mouse antibody genes to human antibody genes to obtain a molecule with appropriate antigen specificity and biological activity, can be used (see, e.g., Morrison S L et al. Proc. Natl. Acad. Sci. 81:6851-6855 (1984); Neuberger M S et al. Nature 312:604-608 (1984); and Takeda S et al. Nature 314:452-454 (1985), both of which are herein incorporated by reference).

Various immunoassays may be used for screening to identify antibodies having the desired specificity. Numerous protocols for competitive binding or immunoradiometric assays using either polyclonal or monoclonal antibodies with established specificities are well known in the art. The antibody can also be a humanized antibody. Antibodies are humanized so that they are less immunogenic and therefore persist longer when administered therapeutically to a patient.

Human antibodies can be generated using the XENOMOUSE technology from Abgenix (Fremont, Calif, USA), which enables the generation and selection of high affinity, fully human antibody product candidates to essentially any disease target appropriate for antibody therapy. See, U.S. Pat. Nos. 6,235,883; 6,207,418; 6,162,963; 6,150,584; 6,130,364; 6,114,598; 6,091,001; 6,075,181; 5,998,209; 5,985,615; 5,939,598; and 5,916,771, each incorporated by reference; Yang X et al., Crit Rev Oncol Hemato 38(1): 17-23 (2001); Chadd H E & Chamow S M. Curr Opin Biotechnol 12(2):188-94 (2001); Green L L, Journal of Immunological Methods 231 11-23 (1999); Yang X-D et al., Cancer Research 59(6): 1236-1243 (1999); and Jakobovits A, Advanced Drug Delivery Reviews 31: 33-42 (1998). Antibodies with fully human protein sequences are generated using genetically engineered strains of mice in which mouse antibody gene expression is suppressed and functionally replaced with human antibody gene expression, while leaving intact the rest of the mouse immune system.

In some embodiments of the present invention, the anti-tumorigenic therapeutic agents (e.g. hedgehog signaling pathway antagonists) of the present invention are co-adminstered with other anti-neoplastic therapies. A wide range of therapeutic agents find use with the present invention. Any therapeutic agent that can be co-administered with the agents of the present invention, or associated with the agents of the present invention is suitable for use in the methods of the present invention.

Some embodiments of the present invention provide methods (therapeutic methods, research methods, drug screening methods) for administering a therapeutic compound of the present invention and at least one additional therapeutic agent (e.g., including, but not limited to, chemotherapeutic antineoplastics, antimicrobials, antivirals, antifungals, and anti-inflammatory agents) and/or therapeutic technique (e.g., surgical intervention, radiotherapies).

Various classes of antineoplastic (e.g., anticancer) agents are contemplated for use in certain embodiments of the present invention. Anticancer agents suitable for use with the present invention include, but are not limited to, agents that induce apoptosis, agents that inhibit adenosine deaminase function, inhibit pyrimidine biosynthesis, inhibit purine ring biosynthesis, inhibit nucleotide interconversions, inhibit ribonucleotide reductase, inhibit thymidine monophosphate (TMP) synthesis, inhibit dihydrofolate reduction, inhibit DNA synthesis, form adducts with DNA, damage DNA, inhibit DNA repair, intercalate with DNA, deaminate asparagines, inhibit RNA synthesis, inhibit protein synthesis or stability, inhibit microtubule synthesis or function, and the like.

In some embodiments, exemplary anticancer agents suitable for use in compositions and methods of the present invention include, but are not limited to: 1) alkaloids, including microtubule inhibitors (e.g., vincristine, vinblastine, and vindesine, etc.), microtubule stabilizers (e.g., paclitaxel (TAXOL), and docetaxel, etc.), and chromatin function inhibitors, including topoisomerase inhibitors, such as epipodophyllotoxins (e.g., etoposide (VP-16), and teniposide (VM-26), etc.), and agents that target topoisomerase I (e.g., camptothecin and isirinotecan (CPT-11), etc.); 2) covalent DNA-binding agents (alkylating agents), including nitrogen mustards (e.g., mechlorethamine, chlorambucil, cyclophosphamide, ifosphamide, and busulfan (MYLERAN), etc.), nitrosoureas (e.g., carmustine, lomustine, and semustine, etc.), and other alkylating agents (e.g., dacarbazine, hydroxymethylmelamine, thiotepa, and mitomycin, etc.); 3) noncovalent DNA-binding agents (antitumor antibiotics), including nucleic acid inhibitors (e.g., dactinomycin (actinomycin D), etc.), anthracyclines (e.g., daunorubicin (daunomycin, and cerubidine), doxorubicin (adriamycin), and idarubicin (idamycin), etc.), anthracenediones (e.g., anthracycline analogues, such as mitoxantrone, etc.), bleomycins (BLENOXANE), etc., and plicamycin (mithramycin), etc.; 4) antimetabolites, including antifolates (e.g., methotrexate, FOLEX, and MEXATE, etc.), purine antimetabolites (e.g., 6-mercaptopurine (6-MP, PURINETHOL), 6-thioguanine (6-TG), azathioprine, acyclovir, ganciclovir, chlorodeoxyadenosine, 2-chlorodeoxyadenosine (CdA), and 2′-deoxycoformycin (pentostatin), etc.), pyrimidine antagonists (e.g., fluoropyrimidines (e.g., 5-fluorouracil (ADRUCIL), 5-fluorodeoxyuridine (FdUrd) (floxuridine)) etc.), and cytosine arabinosides (e.g., CYTOSAR (ara-C) and fludarabine, etc.); 5) enzymes, including L-asparaginase, and hydroxyurea, etc.; 6) hormones, including glucocorticoids, antiestrogens (e.g., tamoxifen, etc.), nonsteroidal antiandrogens (e.g., flutamide, etc.), and aromatase inhibitors (e.g., anastrozole (ARIMIDEX), etc.); 7) platinum compounds (e.g., cisplatin and carboplatin, etc.); 8) monoclonal antibodies conjugated with anticancer drugs, toxins, and/or radionuclides, etc.; 9) biological response modifiers (e.g., interferons (e.g., IFN-α, etc.) and interleukins (e.g., IL-2, etc.), etc.); 10) adoptive immunotherapy; 11) hematopoietic growth factors; 12) agents that induce tumor cell differentiation (e.g., all-trans-retinoic acid, etc.); 13) gene therapy techniques; 14) antisense therapy techniques; 15) tumor vaccines; 16) therapies directed against tumor metastases (e.g., batimastat, etc.); 17) angiogenesis inhibitors; 18) proteosome inhibitors (e.g., VELCADE); 19) inhibitors of acetylation and/or methylation (e.g., HDAC inhibitors); 20) modulators of NF kappa B; 21) inhibitors of cell cycle regulation (e.g., CDK inhibitors); 22) modulators of p53 protein function; and 23) radiation.

Any oncolytic agent that is routinely used in a cancer therapy context finds use in the compositions and methods of the present invention. For example, the U.S. Food and Drug Administration maintains a formulary of oncolytic agents approved for use in the United States. International counterpart agencies to the U.S.F.D.A. maintain similar formularies. Table 1 provides a list of exemplary antineoplastic agents approved for use in the U.S. Those skilled in the art will appreciate that the “product labels” required on all U.S. approved chemotherapeutics describe approved indications, dosing information, toxicity data, and the like, for the exemplary agents.

TABLE 1

| | | |
|---|---|---|
| Aldesleukin | Proleukin | Chiron Corp., |
| (des-alanyl-1, serine-125 human interleukin-2) | | Emeryville, CA |
| Alemtuzumab | Campath | Millennium and ILEX |
| (IgG1κ anti CD52 antibody) | | Partners, LP, Cambridge, MA |
| Alitretinoin | Panretin | Ligand Pharmaceuticals, |
| (9-cis-retinoic acid) | | Inc., San Diego CA |
| Allopurinol | Zyloprim | GlaxoSmithKline, |
| (1,5-dihydro-4 H -pyrazolo[3,4-d]pyrimidin-4-one | | Research Triangle |
| monosodium salt) | | Park, NC |
| Altretamine | Hexalen | US Bioscience, West |
| (N,N,N′,N′,N″,N″,-hexamethyl-1,3,5-triazine-2, 4, | | Conshohocken, PA |
| 6-triamine) | | |
| Amifostine | Ethyol | US Bioscience |
| (ethanethiol, 2-[(3-aminopropyl)amino]-, | | |
| dihydrogen phosphate (ester)) | | |
| Anastrozole | Arimidex | AstraZeneca |
| (1,3-Benzenediacetonitrile, a, a, a′, a′-tetramethyl- | | Pharmaceuticals, LP, |
| 5-(1H-1,2,4-triazol-1-ylmethyl)) | | Wilmington, DE |
| Arsenic trioxide | Trisenox | Cell Therapeutic, Inc., |
| | | Seattle, WA |
| Asparaginase | Elspar | Merck & Co., Inc., |
| (L-asparagine amidohydrolase, type EC-2) | | Whitehouse Station, NJ |
| BCG Live | TICE BCG | Organon Teknika, |
| (lyophilized preparation of an attenuated strain of | | Corp., Durham, NC |
| Mycobacterium bovis (Bacillus Calmette-Gukin | | |
| [BCG], substrain Montreal) | | |
| bexarotene capsules | Targretin | Ligand |
| (4-[1-(5,6,7,8-tetrahydro-3,5,5,8,8-pentamethyl-2- | | Pharmaceuticals |
| napthalenyl) ethenyl] benzoic acid) | | |
| bexarotene gel | Targretin | Ligand |
| | | Pharmaceuticals |
| Bleomycin | Blenoxane | Bristol-Myers Squibb |
| (cytotoxic glycopeptide antibiotics produced by | | Co., NY, NY |
| Streptomyces verticillus; bleomycin A₂ and | | |
| bleomycin B₂) | | |
| Capecitabine | Xeloda | Roche |
| (5′-deoxy-5-fluoro-N-[(pentyloxy)carbonyl]- | | |
| cytidine) | | |
| Carboplatin | Paraplatin | Bristol-Myers Squibb |
| (platinum, diammine [1,1- | | |
| cyclobutanedicarboxylato(2-)-0, 0′]-,(SP-4-2)) | | |
| Carmustine | BCNU, BiCNU | Bristol-Myers Squibb |
| (1,3-bis(2-chloroethyl)-1-nitrosourea) | | |
| Carmustine with Polifeprosan 20 Implant | Gliadel Wafer | Guilford Pharmaceuticals, |
| | | Inc., Baltimore, MD |
| Celecoxib | Celebrex | Searle |
| (as 4-[5-(4-methylphenyl)-3- (trifluoromethyl)-1H- | | Pharmaceuticals, |
| pyrazol-1-yl]benzenesulfonamide) | | England |
| Chlorambucil | Leukeran | GlaxoSmithKline |
| (4-[bis(2chlorethyl)amino]benzenebutanoic acid) | | |
| Cisplatin | Platinol | Bristol-Myers Squibb |
| (PtCl₂H₆N₂) | | |
| Cladribine | Leustatin, 2-CdA | R. W. Johnson Pharmaceutical |
| (2-chloro-2′-deoxy-b-D-adenosine) | | Research Institute, |
| | | Raritan, NJ |
| Cyclophosphamide | Cytoxan, Neosar | Bristol-Myers Squibb |
| (2-[bis(2-chloroethyl)amino] tetrahydro-2H-13,2- | | |
| oxazaphosphorine 2-oxide monohydrate) | | |
| Cytarabine | Cytosar-U | Pharmacia & Upjohn |
| (1-b-D-Arabinofuranosylcytosine, C₉H₁₃N₃O₅) | | Company |
| cytarabine liposomal | DepoCyt | Skye Pharmaceuticals, |
| | | Inc., San Diego, CA |
| Dacarbazine | DTIC-Dome | Bayer AG, |
| (5-(3,3-dimethyl-l-triazeno)-imidazole-4- | | Leverkusen, Germany |
| carboxamide (DTIC)) | | |
| Dactinomycin, actinomycin D | Cosmegen | Merck |
| (actinomycin produced by Streptomyces parvullus, | | |
| C₆₂H₈₆N₁₂O₁₆) | | |
| Darbepoetin alfa | Aranesp | Amgen, Inc., |
| (recombinant peptide) | | Thousand Oaks, CA |
| daunorubicin liposomal | DanuoXome | Nexstar |
| ((8S-cis)-8-acetyl-10-[(3-amino-2,3,6-trideoxy-á- | | Pharmaceuticals, Inc., |
| L-lyxo-hexopyranosyl)oxy]-7,8,9,10-tetrahydro-6,8,11- | | Boulder, CO |
| trihydroxy-1-methoxy-5,12-naphthacenedione hydrochloride) | | |
| Daunorubicin HCl, daunomycin | Cerubidine | Wyeth Ayerst, |
| ((1 S ,3 S)-3-Acetyl-1,2,3,4,6,11-hexahydro-3,5,12- | | Madison, NJ |
| trihydroxy-10-methoxy-6,11-dioxo-1-naphthacenyl 3-amino- | | |
| 2,3,6-trideoxy-(alpha)-L-lyxo-hexopyranoside hydrochloride) | | |
| Denileukin diftitox | Ontak | Seragen, Inc., |
| (recombinant peptide) | | Hopkinton, MA |
| Dexrazoxane | Zinecard | Pharmacia & Upjohn |
| ((S)-4,4′-(1-methyl-1,2-ethanediyl)bis-2,6- | | Company |
| piperazinedione) | | |
| Docetaxel | Taxotere | Aventis |
| ((2R,3S)-N-carboxy-3-phenylisoserine, N-tert-butyl ester, | | Pharmaceuticals, Inc., |
| 13-ester with 5b-20-epoxy-12a,4,7b,10b,13a- | | Bridgewater, NJ |
| hexahydroxytax- 11-en-9-one 4-acetate 2-benzoate, trihydrate) | | |
| Doxorubicin HCl | Adriamycin, | Pharmacia & Upjohn |
| (8S,10S)-10-[(3-amino-2,3,6-trideoxy-a-L-lyxo- | Rubex | Company |
| hexopyranosyl)oxy] -8-glycolyl-7,8,9,10-tetrahydro-6,8,11- | | |
| trihydroxy-1-methoxy-5,12-naphthacenedione hydrochloride) | | |
| doxorubicin | Adriamycin PFS | Pharmacia & Upjohn |
| | Intravenous injection | Company |
| doxorubicin liposomal | Doxil | Sequus Pharmaceuticals, |
| | | Inc., Menlo park, CA |
| dromostanolone propionate | Dromostanolone | Eli Lilly & Company, |
| (17b-Hydroxy-2a-methyl-5a-androstan-3-one | | Indianapolis, IN |
| propionate) | | |
| dromostanolone propionate | Masterone | Syntex, Corp., Palo |
| | injection | Alto, CA |
| Elliott's B Solution | Elliott's B | Orphan Medical, Inc |
| | Solution | |
| Epirubicin | Ellence | Pharmacia & Upjohn |
| ((8S-cis)-10-[(3-amino-2,3,6-trideoxy-a-L-arabino- | | Company |
| hexopyranosyl)oxy]-7,8,9,10-tetrahydro-6,8,11-trihydroxy-8- | | |
| (hydroxyacetyl)-1-methoxy-5,12-naphthacenedione hydrochloride) | | |
| Epoetin alfa | Epogen | Amgen, Inc |
| (recombinant peptide) | | |
| Estramustine | Emcyt | Pharmacia & Upjohn |
| (estra-1,3,5(10)-triene-3,17-diol(17(beta))-, 3-[bis(2- | | Company |
| chloroethyl)carbamate]17-(dihydrogen phosphate), | | |
| disodium salt, monohydrate, or estradiol 3-[bis(2- | | |
| chloroethyl)carbamate] 17-(dihydrogen phosphate), disodium | | |
| salt, monohydrate) | | |
| Etoposide phosphate | Etopophos | Bristol-Myers Squibb |
| (4′-Demethylepipodophyllotoxin 9-[4,6-O—R)- | | |
| ethylidene-(beta)-D-glucopyranoside], 4′- | | |
| (dihydrogen phosphate)) | | |
| etoposide, VP-16 | Vepesid | Bristol-Myers Squibb |
| (4′-demethylepipodophyllotoxin 9-[4,6-0-(R)- | | |
| ethylidene-(beta)-D-glucopyranoside]) | | |
| Exemestane | Aromasin | Pharmacia & Upjohn |
| (6-methylenandrosta-1,4-diene-3, 17-dione) | | Company |
| Filgrastim | Neupogen | Amgen, Inc |
| (r-metHuG-CSF) | | |
| floxuridine (intraarterial) | FUDR | Roche |
| (2′-deoxy-5-fluorouridine) | | |
| Fludarabine | Fludara | Berlex Laboratories, |
| (fluorinated nucleotide analog of the antiviral agent | | Inc., Cedar Knolls, NJ |
| vidarabine, 9-b -D-arabinofuranosyladenine(ara-A)) | | |
| Fluorouracil, 5-FU | Adrucil | ICN Pharmaceuticals, |
| (5-fluoro-2,4(1H,3H)-pyrimidinedione) | | Inc., Humacao, Puerto Rico |
| Fulvestrant | Faslodex | IPR Pharmaceuticals, |
| (7-alpha-[9-(4,4,5,5,5-penta fluoropentylsulphinyl) | | Guayama, Puerto Rico |
| nonyl]estra-1,3,5-(10)- triene-3,17-beta-diol) | | |
| Gemcitabine | Gemzar | Eli Lilly |
| (2′-deoxy-2′, 2′-difluorocytidine | | |
| monohydrochloride (b-isomer)) | | |
| Gemtuzumab Ozogamicin | Mylotarg | Wyeth Ayerst |
| (anti-CD33 hP67.6) | | |
| Goserelin acetate | Zoladex Implant | AstraZeneca |
| (acetate salt of [D-Ser(But)⁶,Azgly¹⁰]LHRH; pyro- | | Pharmaceuticals |
| Glu-His-Trp-Ser-Tyr-D-Ser(But)-Leu-Arg-Pro- | | |
| Azgly-NH2 acetate [C₅₉H₈₄N₁₈O₁₄ •(C₂H₄O₂)ₓ | | |
| Hydroxyurea | Hydrea | Bristol-Myers Squibb |
| Ibritumomab Tiuxetan | Zevalin | Biogen IDEC, Inc., |
| (immunoconjugate resulting from a thiourea | | Cambridge MA |
| covalent bond between the monoclonal antibody | | |
| Ibritumomab and the linker-chelator tiuxetan [N- | | |
| [2-bis(carboxymethyl)amino]-3-(p-isothiocyanatophenyl)- | | |
| propyl]-[N-[2-bis(carboxymethyl)amino]-2-(methyl) - | | |
| ethyl]glycine) | | |
| Idarubicin | Idamycin | Pharmacia & Upjohn |
| (5, 12-Naphthacenedione, 9-acetyl-7-[(3-amino- | | Company |
| 2,3,6-trideoxy-(alpha)-L- lyxo - hexopyranosyl)oxy]- | | |
| 7,8,9,10-tetrahydro-6,9,11-trihydroxyhydrochloride, (7S- cis)) | | |
| Ifosfamide | IFEX | Bristol-Myers Squibb |
| (3-(2-chloroethyl)-2-[(2- | | |
| chloroethyl)amino]tetrahydro-2H-1,3,2- | | |
| oxazaphosphorine 2-oxide) | | |
| Imatinib Mesilate | Gleevec | Novartis AG, Basel, |
| (4-[(4-Methyl-1-piperazinyl)methyl]-N-[4-methyl- | | Switzerland |
| 3-[[4-(3-pyridinyl)-2-pyrimidinyl]amino]- | | |
| phenyl]benzamide methanesulfonate) | | |
| Interferon alfa-2a | Roferon-A | Hoffmann-La Roche, |
| (recombinant peptide) | | Inc., Nutley, NJ |
| Interferon alfa-2b | Intron A | Schering AG, Berlin, |
| (recombinant peptide) | (Lyophilized | Germany |
| | Betaseron) | |
| Irinotecan HCl | Camptosar | Pharmacia & Upjohn |
| ((4S)-4,1 1-diethyl-4-hydroxy-9-[(4- piperi- | | Company |
| dinopiperidino)carbonyloxy]-1H-pyrano[3′, 4′: 6,7] | | |
| indolizino[1,2-b] quinoline-3,14(4H, 12H) dione | | |
| hydrochloride trihydrate) | | |
| Letrozole | Femara | Novartis |
| (4,4′-(1H-1,2,4 - Triazol-1-ylmethylene) | | |
| dibenzonitrile) | | |
| Leucovorin | Wellcovorin, | Immunex, Corp., |
| (L-Glutamic acid, N[4[[(2amino-5-formyl- | Leucovorin | Seattle, WA |
| 1,4,5,6,7,8 hexahydro4oxo6- | | |
| pteridinyl)methyl]amino]benzoyl], calcium salt | | |
| (1:1)) | | |
| Levamisole HCl | Ergamisol | Janssen Research |
| ((−)-(S)-2,3,5, 6-tetrahydro-6-phenylimidazo [2,1- | | Foundation, |
| b] thiazole monohydrochloride C₁₁H₁₂N₂S•HCl) | | Titusville, NJ |
| Lomustine | CeeNU | Bristol-Myers Squibb |
| (1-(2-chloro-ethyl)-3-cyclohexyl-1-nitrosourea) | | |
| Meclorethamine, nitrogen mustard | Mustargen | Merck |
| (2-chloro-N-(2-chloroethyl)-N-methylethanamine | | |
| hydrochloride) | | |
| Megestrol acetate | Megace | Bristol-Myers Squibb |
| 17α( acetyloxy)- 6- methylpregna- 4,6- diene-3,20- dione | | |
| Melphalan, L-PAM | Alkeran | GlaxoSmithKline |
| (4-[bis(2-chloroethyl) amino]-L-phenylalanine) | | |
| Mercaptopurine, 6-MP | Purinethol | GlaxoSmithKline |
| (1,7-dihydro-6 H -purine-6-thione monohydrate) | | |
| Mesna | Mesnex | Asta Medica |
| [sodium 2-mercaptoethane sulfonate) | | |
| Methotrexate | Methotrexate | Lederle Laboratories |
| (N-[4-[[(2,4-diamino-6- | | |
| pteridinyl)methyl]methylamino]benzoyl]-L- | | |
| glutamic acid) | | |
| Methoxsalen | Uvadex | Therakos, Inc., Way |
| [9-methoxy-7H-furo[3,2-g][1]-benzopyran-7-one) | | Exton, Pa |
| Mitomycin C | Mutamycin | Bristol-Myers Squibb |
| mitomycin C | Mitozytrex | SuperGen, Inc., Dublin, CA |
| Mitotane | Lysodren | Bristol-Myers Squibb |
| (1,1-dichloro-2-(o-chlorophenyl)-2-(p-chlorophenyl) ethane) | | |
| Mitoxantrone | Novantrone | Immunex Corporation |
| (1,4-dihydroxy-5,8-bis[[2- [(2- | | |
| hydroxyethyl)amino]ethyl]amino]-9,10- | | |
| anthracenedione dihydrochloride) | | |
| Nandrolone phenpropionate | Durabolin-50 | Organon, Inc., West Orange, NJ |
| Nofetumomab | Verluma | Boehringer Ingelheim |
| | | Pharma KG, Germany |
| Oprelvekin | Neumega | Genetics Institute, |
| (IL-11) | | Inc., Alexandria, VA |
| Oxaliplatin | Eloxatin | Sanofi Synthelabo, |
| (cis-[(1R,2R)-1,2-cyclohexanediamine-N,N′] | | Inc., NY, NY |
| [oxalato(2-)-O,0′] platinum) | | |
| Paclitaxel | TAXOL | Bristol-Myers Squibb |
| (5β, 20-Epoxy-1,2a, 4,7β, 10β, 13a- | | |
| hexahydroxytax-11-en-9-one 4,10-diacetate 2- | | |
| benzoate 13-ester with (2R, 3 S)- N-benzoyl-3- | | |
| phenylisoserine) | | |
| Pamidronate | Aredia | Novartis |
| (phosphonic acid (3-amino-1-hydroxypropylidene) | | |
| bis-, disodium salt, pentahydrate, (APD)) | | |
| Pegademase | Adagen | Enzon |
| ((monomethoxypolyethylene glycol succinimidyl) | (Pegademase | Pharmaceuticals, Inc., |
| 11 - 17 -adenosine deaminase) | Bovine) | Bridgewater, NJ |
| Pegaspargase | Oncaspar | Enzon |
| (monomethoxypolyethylene glycol succinimidyl L- | | |
| asparaginase) | | |
| Pegfilgrastim | Neulasta | Amgen, Inc |
| (covalent conjugate of recombinant methionyl human | | |
| G-CSF (Filgrastim) and monomethoxypolyethylene glycol) | | |
| Pentostatin | Nipent | Parke-Davis Pharmaceutical |
| | | Co., Rockville, MD |
| Pipobroman | Vercyte | Abbott Laboratories, |
| | | Abbott Park, IL |
| Plicamycin, Mithramycin | Mithracin | Pfizer, Inc., NY, NY |
| (antibiotic produced by Streptomyces plicatus) | | |
| Porfimer sodium | Photofrin | QLT Phototherapeutics, |
| | | Inc., Vancouver, Canada |
| Procarbazine | Matulane | Sigma Tau |
| (N-isopropyl-μ-(2-methylhydrazino)-p-toluamide | | Pharmaceuticals, Inc., |
| monohydrochloride) | | Gaithersburg, MD |
| Quinacrine | Atabrine | Abbott Labs |
| (6-chloro-9-( 1 -methyl-4-diethyl-amine) | | |
| butylamino-2-methoxyacridine) | | |
| Rasburicase | Elitek | Sanofi-Synthelabo, |
| (recombinant peptide) | | Inc., |
| Rituximab | Rituxan | Genentech, Inc., |
| (recombinant anti-CD20 antibody) | | South San Francisco, CA |
| Sargramostim | Prokine | Immunex Corp |
| (recombinant peptide) | | |
| Streptozocin | Zanosar | Pharmacia & Upjohn |
| (streptozocin 2 -deoxy - 2 - | | Company |
| [[(methylnitrosoamino)carbonyl]amino] - a(and b )- | | |
| D - glucopyranose and 220 mg citric acid anhydrous) | | |
| Talc | Sclerosol | Bryan, Corp., |
| (Mg₃Si₄O₁₀ (OH)₂) | | Woburn, MA |
| Tamoxifen | Nolvadex | AstraZeneca |
| ((Z)2-[4-(1,2-diphenyl-1-butenyl) phenoxy]-N, N- | | Pharmaceuticals |
| dimethylethanamine 2-hydroxy-1,2,3-propanetricarboxylate (1:1)) | | |
| Temozolomide | Temodar | Schering |
| (3,4-dihydro-3-methyl-4-oxoimidazo[5,1-d]-as- | | |
| tetrazine-8-carboxamide) | | |
| teniposide, VM-26 | Vumon | Bristol-Myers Squibb |
| (4′-demethylepipodophyllotoxin 9-[4,6-0-(R)-2- | | |
| thenylidene-(beta)-D-glucopyranoside]) | | |
| Testolactone | Teslac | Bristol-Myers Squibb |
| (13-hydroxy-3-oxo-13,17-secoandrosta-1,4-dien- | | |
| 17-oic acid [dgr]-lactone) | | |
| Thioguanine, 6-TG | Thioguanine | GlaxoSmithKline |
| (2-amino-1,7-dihydro-6 H - purine-6-thione) | | |
| Thiotepa | Thioplex | Immunex Corporation |
| (Aziridine, 1,1′,1″-phosphinothioylidynetris-, or | | |
| Tris (1-aziridinyl) phosphine sulfide) | | |
| Topotecan HCl | Hycamtin | GlaxoSmithKline |
| ((S)-10-[(dimethylamino) methyl]-4-ethyl-4,9- | | |
| dihydroxy-1H-pyrano[3′, 4′: 6,7] indolizino [1,2-b] | | |
| quinoline-3,14-(4H,12H)-dione monohydrochloride) | | |
| Toremifene | Fareston | Roberts |
| (2-(p-[(Z)-4-chloro-1,2-diphenyl-1-butenyl]- | | Pharmaceutical Corp., |
| phenoxy)-N,N-dimethylethylamine citrate (1:1)) | | Eatontown, NJ |
| Tositumomab, I 131 Tositumomab | Bexxar | Corixa Corp., Seattle, |
| (recombinant murine immunotherapeutic | | WA |
| monoclonal IgG₂ₐ lambda anti-CD20 antibody (I | | |
| 131 is a radioimmunotherapeutic antibody)) | | |
| Trastuzumab | Herceptin | Genentech, Inc |
| (recombinant monoclonal IgG₁ kappa anti-HER2 antibody) | | |
| Tretinoin, ATRA | Vesanoid | Roche |
| (all-trans retinoic acid) | | |
| Uracil Mustard | Uracil Mustard Capsules | Roberts Labs |
| Valrubicin, N-trifluoroacetyladriamycin-14-valerate | Valstar | Anthra --> Medeva |
| ((2S-cis)-2-[1,2,3,4,6,11-hexahydro-2,5,12-trihydroxy- | | |
| 7 methoxy-6,11-dioxo-[[4 2,3,6-trideoxy-3- | | |
| [(trifluoroacetyl)-amino-α-L-lyxo-hexopyranosyl]oxyl]- | | |
| 2-naphthacenyl]-2-oxoethyl pentanoate) | | |
| Vinblastine, Leurocristine | Velban | Eli Lilly |
| (C₄₆H₅₆N₄O₁₀•H₂SO₄) | | |
| Vincristine | Oncovin | Eli Lilly |
| (C₄₆H₅₆N₄O₁₀•H₂SO₄) | | |
| Vinorelbine | Navelbine | GlaxoSmithKline |
| (3′ ,4′-didehydro-4′-deoxy-C′-norvincaleukoblastine | | |
| [R-(R*,R*)-2,3-dihydroxybutanedioate (1:2)(salt)]) | | |
| Zoledronate, Zoledronic acid | Zometa | Novartis |
| ((1-Hydroxy-2-imidazol-1-yl-phosphonoethyl) | | |
| phosphonic acid monohydrate) | | |

Antimicrobial therapeutic agents may also be used as therapeutic agents in the present invention. Any agent that can kill, inhibit, or otherwise attenuate the function of microbial organisms may be used, as well as any agent contemplated to have such activities. Antimicrobial agents include, but are not limited to, natural and synthetic antibiotics, antibodies, inhibitory proteins (e.g., defensins), antisense nucleic acids, membrane disruptive agents and the like, used alone or in combination. Indeed, any type of antibiotic may be used including, but not limited to, antibacterial agents, antiviral agents, antifungal agents, and the like.

In still further embodiments, the present invention provides compounds of the present invention (and any other chemotherapeutic agents) associated with targeting agents that are able to specifically target particular cell types (e.g., tumor cells). Generally, the therapeutic compound that is associated with a targeting agent, targets neoplastic cells through interaction of the targeting agent with a cell surface moiety that is taken into the cell through receptor mediated endocytosis.

Any moiety known to be located on the surface of target cells (e.g., tumor cells) finds use with the present invention. For example, an antibody directed against such a moiety targets the compositions of the present invention to cell surfaces containing the moiety. Alternatively, the targeting moiety may be a ligand directed to a receptor present on the cell surface or vice versa. Similarly, vitamins also may be used to target the therapeutics of the present invention to a particular cell.

As used herein, the term “targeting molecules” refers to chemical moieties, and portions thereof useful for targeting therapeutic compounds to cells, tissues, and organs of interest. Various types of targeting molecules are contemplated for use with the present invention including, but not limited to, signal peptides, antibodies, nucleic acids, toxins and the like. Targeting moieties may additionally promote the binding of the associated chemical compounds (e.g., small molecules) or the entry of the compounds into the targeted cells, tissues, and organs. Preferably, targeting moieties are selected according to their specificity, affinity, and efficacy in selectively delivering attached compounds to targeted sites within a subject, tissue, or a cell, including specific subcellular locations and organelles.

Various efficiency issues affect the administration of all drugs--and of highly cytotoxic drugs (e.g., anticancer drugs) in particular. One issue of particular importance is ensuring that the administered agents affect only targeted cells (e.g., cancer cells), tissues, or organs. The nonspecific or unintended delivery of highly cytotoxic agents to nontargeted cells can cause serious toxicity issues.

Numerous attempts have been made to devise drug-targeting schemes to address the problems associated with nonspecific drug delivery. (See e.g., K. N. Syrigos and A. A. Epenetos Anticancer Res., 19:606-614 (1999); Y. J. Park et al., J. Controlled Release, 78:67-79 (2002); R. V. J. Chari, Adv. Drug Deliv. Rev., 31:89-104 (1998); and D. Putnam and J. Kopecek, Adv. Polymer Sci., 122:55-123 (1995)). Conjugating targeting moieties such as antibodies and ligand peptides (e.g., RDG for endothelium cells) to drug molecules has been used to alleviate some collateral toxicity issues associated with particular drugs.

The compounds and anticancer agents may be administered in any sterile, biocompatible pharmaceutical carrier, including, but not limited to, saline, buffered saline, dextrose, and water. In some embodiments, the pharmaceutical compositions of the present invention may contain one agent (e.g., an antibody). In other embodiments, the pharmaceutical compositions contain a mixture of at least two agents (e.g., an antibody and one or more conventional anticancer agents). In still further embodiments, the pharmaceutical compositions of the present invention contain at least two agents that are administered to a patient under one or more of the following conditions: at different periodicities, at different durations, at different concentrations, by different administration routes, etc. In some embodiments, the hedgehog signaling pathway antagonist is administered prior to the second anticancer agent, e.g., 0.5, 1, 2 3, 4, 5, 10, 12, or 18 hours, 1, 2, 3, 4, 5, or 6 days, 1, 2, 3, or 4 weeks prior to the administration of the anticancer agent. In some embodiments, the hedgehog signaling pathway antagonist is administered after the second anticancer agent, e.g., 0.5, 1, 2 3, 4, 5, 10, 12, or 18 hours, 1, 2, 3, 4, 5, or 6 days, 1, 2, 3, or 4 weeks after the administration of the anticancer agent. In some embodiments, the hedgehog signaling pathway antagonist and the second anticancer agent are administered concurrently but on different schedules, e.g., the hedgehog signaling pathway antagonist compound is administered daily while the second anticancer agent is administered once a week, once every two weeks, once every three weeks, or once every four weeks. In other embodiments, the hedgehog signaling pathway antagonist is administered once a week while the second anticancer agent is administered daily, once a week, once every two weeks, once every three weeks, or once every four weeks.

Depending on the condition being treated, preferred embodiments of the present pharmaceutical compositions are formulated and administered systemically or locally. Techniques for formulation and administration can be found in the latest edition of “Remington's Pharmaceutical Sciences” (Mack Publishing Co, Easton Pa.). Suitable routes may, for example, include oral or transmucosal administration as well as parenteral delivery (e.g., intramuscular, subcutaneous, intramedullary, intrathecal, intraventricular, intravenous, intraperitoneal, or intranasal administration).

The present invention contemplates administering therapeutic compounds and, in some embodiments, one or more conventional anticancer agents, in accordance with acceptable pharmaceutical delivery methods and preparation techniques. For example, therapeutic compounds and suitable anticancer agents can be administered to a subject intravenously in a pharmaceutically acceptable carrier such as physiological saline. Standard methods for intracellular delivery of pharmaceutical agents are contemplated (e.g., delivery via liposome). Such methods are well known to those of ordinary skill in the art.

In some embodiments, the formulations of the present invention are useful for parenteral administration (e.g., intravenous, subcutaneous, intramuscular, intramedullary, and intraperitoneal). Therapeutic co-administration of some contemplated anticancer agents (e.g., therapeutic polypeptides) can also be accomplished using gene therapy reagents and techniques.

In some embodiments of the present invention, therapeutic compounds are administered to a subject alone, or in combination with one or more conventional anticancer agents (e.g., nucleotide sequences, drugs, hormones, etc.) or in pharmaceutical compositions where the components are optionally mixed with excipient(s) or other pharmaceutically acceptable carriers. In preferred embodiments of the present invention, pharmaceutically acceptable carriers are biologically inert. In preferred embodiments, the pharmaceutical compositions of the present invention are formulated using pharmaceutically acceptable carriers well known in the art in dosages suitable for oral administration. Such carriers enable the pharmaceutical compositions to be formulated as tablets, pills, capsules, dragees, liquids, gels, syrups, slurries, solutions, suspensions and the like, for respective oral or nasal ingestion by a subject.

Pharmaceutical preparations for oral use can be obtained by combining the active compounds with solid excipients, optionally grinding the resulting mixture, and processing the mixture into granules, after adding suitable auxiliaries, if desired, to obtain tablets or dragee cores. Suitable excipients are carbohydrate or protein fillers such as sugars, including lactose, sucrose, mannitol, or sorbitol; starch from corn, wheat, rice, potato, etc.; cellulose such as methyl cellulose, hydroxypropylmethyl-cellulose, or sodium carboxymethylcellulose; gums including arabic and tragacanth; and proteins such as gelatin and collagen. If desired, disintegrating or solubilizing agents may be added, such as the cross-linked polyvinyl pyrrolidone, agar, alginic acid or a salt thereof such as sodium alginate.

In preferred embodiments, dosing and administration regimes are tailored by the clinician, or others skilled in the pharmacological arts, based upon well known pharmacological and therapeutic considerations including, but not limited to, the desired level of therapeutic effect, and the practical level of therapeutic effect obtainable. Generally, it is advisable to follow well-known pharmacological principles for administrating chemotherapeutic agents (e.g., it is generally advisable to not change dosages by more than 50% at time and no more than every 3-4 agent half-lives). For compositions that have relatively little or no dose-related toxicity considerations, and where maximum efficacy (e.g., destruction of cancer cells) is desired, doses in excess of the average required dose are not uncommon. This approach to dosing is commonly referred to as the “maximal dose” strategy. In certain embodiments, the hedgehog signaling pathway antagonist is administered to a subject at a dose of 1-40 mg per day (e.g. for 4-6 weeks). In certain embodiments, subject is administered a loading dose of between 15-70 mg of the hedgehog signaling pathway antagonist. In certain embodiments, the subject is administered a loading dose of about 35-45 mg of the hedgehog signaling pathway antagonist (e.g. subcutaneously), and then daily doses of about 10 mg (e.g. subcutaneously) for about 4-6 weeks.

Additional dosing considerations relate to calculating proper target levels for the agent being administered, the agent's accumulation and potential toxicity, stimulation of resistance, lack of efficacy, and describing the range of the agent's therapeutic index.

In certain embodiments, the present invention contemplates using routine methods of titrating the agent's administration. One common strategy for the administration is to set a reasonable target level for the agent in the subject. In some preferred embodiments, agent levels are measured in the subject's plasma. Proper dose levels and frequencies are then designed to achieve the desired steady-state target level for the agent. Actual, or average, levels of the agent in the subject are monitored (e.g., hourly, daily, weekly, etc.) such that the dosing levels or frequencies can be adjusted to maintain target levels. Of course, the pharmacokinetics and pharmacodynamics (e.g., bioavailability, clearance or bioaccumulation, biodistribution, drug interactions, etc.) of the particular agent or agents being administered can potentially impact what are considered reasonable target levels and thus impact dosing levels or frequencies.

Target-level dosing methods typically rely upon establishing a reasonable therapeutic objective defined in terms of a desirable range (or therapeutic range) for the agent in the subject. In general, the lower limit of the therapeutic range is roughly equal to the concentration of the agent that provides about 50% of the maximum possible therapeutic effect. The upper limit of the therapeutic range is usually established by the agent's toxicity and not by its efficacy. The present invention contemplates that the upper limit of the therapeutic range for a particular agent will be the concentration at which less than 5 or 10% of subjects exhibit toxic side effects. hi some embodiments, the upper limit of the therapeutic range is about two times, or less, than the lower limit. Those skilled in the art will understand that these dosing consideration are highly variable and to some extent individualistic (e.g., based on genetic predispositions, immunological considerations, tolerances, resistances, and the like). Thus, in some embodiments, effective target dosing levels for an agent in a particular subject may be 1, . . . 5, . . . 10, . . . 15, . . . 20, . . . 50, . . . 75, . . . 100, . . . 200, . . . X %, greater than optimal in another subject. Conversely, some subjects may suffer significant side effects and toxicity related health issues at dosing levels or frequencies far less (1, . . . 5, . . . 10, . . . 15, . . . 20, . . . 50, . . . 75, . . . 100, . . . 200, . . . X %) than those typically producing optimal therapeutic levels in some or a majority of subjects. In the absence of more specific information, target administration levels are often set in the middle of the therapeutic range.

In preferred embodiments, the clinician rationally designs an individualized dosing regimen based on known pharmacological principles and equations. In general, the clinician designs an individualized dosing regimen based on knowledge of various pharmacological and pharmacokinetic properties of the agent, including, but not limited to, F (fractional bioavailability of the dose), Cp (concentration in the plasma), CL (clearance/clearance rate), Vss (volume of drug distribution at steady state) Css (concentration at steady state), and t½ (drug half-life), as well as information about the agent's rate of absorption and distribution. Those skilled in the art are referred to any number of well known pharmacological texts (e.g., Goodman and Gilman's, Pharmaceutical Basis of Therapeutics, 10th ed., Hardman et aL., eds., 2001) for further explanation of these variables and for complete equations illustrating the calculation of individualized dosing regimes. Those skilled in the art also will be able to anticipate potential fluctuations in these variables in individual subjects. For example, the standard deviation in the values observed for F, CL, and Vss is typically about 20%, 50%, and 30%, respectively. The practical effect of potentially widely varying parameters in individual subjects is that 95% of the time the Css achieved in a subject is between 35 and 270% that of the target level. For drugs with low therapeutic indices, this is an undesirably wide range. Those skilled in the art will appreciate, however, that once the agent's Cp (concentration in the plasma) is measured, it is possible to estimate the values of F, CL, and Vss directly. This allows the clinician to effectively fine tune a particular subject's dosing regimen.

In still other embodiments, the present invention contemplates that continuing therapeutic drug monitoring techniques be used to further adjust an individual's dosing methods and regimens. For example, in one embodiment, Css data is used is to further refine the estimates of CL/F and to subsequently adjust the individual's maintenance dosing to achieve desired agent target levels using known pharmacological principles and equations. Therapeutic drug monitoring can be conducted at practically any time during the dosing schedule. In preferred embodiments, monitoring is carried out at multiple time points during dosing and especially when administering intermittent doses. For example, drug monitoring can be conducted concomitantly, within fractions of a second, seconds, minutes, hours, days, weeks, months, etc., of administration of the agent regardless of the dosing methodology employed (e.g., intermittent dosing, loading doses, maintenance dosing, random dosing, or any other dosing method). However, those skilled in the art will appreciate that when sampling rapidly follows agent administration the changes in agent effects and dynamics may not be readily observable because changes in plasma concentration of the agent may be delayed (e.g., due to a slow rate of distribution or other pharmacodynamic factors). Accordingly, subject samples obtained shortly after agent administration may have limited or decreased value.

The primary goal of collecting biological samples from the subject during the predicted steady-state target level of administration is to modify the individual's dosing regimen based upon subsequently calculating revised estimates of the agent's CL/F ratio. However, those skilled in the art will appreciate that early postabsorptive drug concentrations do not typically reflect agent clearance. Early postabsorptive drug concentrations are dictated principally by the agent's rate of absorption, the central, rather than the steady state, volume of agent distribution, and the rate of distribution. Each of these pharmacokinetic characteristics have limited value when calculating therapeutic long-term maintenance dosing regimens.

Accordingly, in some embodiments, when the objective is therapeutic long-term maintenance dosing, biological samples are obtained from the subject, cells, or tissues of interest well after the previous dose has been administered, and even more preferably shortly before the next planned dose is administered.

In still other embodiments, where the therapeutic agent is nearly completely cleared by the subject in the interval between doses, then the present invention contemplates collecting biological samples from the subject at various time points following the previous administration, and most preferably shortly after the dose was administered.

### EXAMPLES

The following example is provided in order to demonstrate and further illustrate certain preferred embodiments and aspects of the present invention and is not to be construed as limiting the scope thereof.

### Example 1

Hedgehog Agonist and Antagonist Treatment of Progenitor Mammary Cells

This example describes methods of assaying the impact of hedgehog agonists and antagonists on cultured progenitor mammary cells.

Materials and Methods

Dissociation of Mammary Tissue and Mammosphere Culture

100-200 gram of normal breast tissue from reduction mammoplasties was minced with scalpels in sterile petri dishes, and transferred to a tissue dissociation flask with 150-300 ml of 300.U/ml collagenase type 3 (Worthington Biochemical Corporation, Lakewood, N.J.) and dissociated approximately 16 hours on a rotary shaker at 37° C. Dissociated tissue was centrifuged for 30 seconds at 40×g in 50 ml centrifuged tubes and the pellets, which were highly enriched with epithelial organoids, was washed several times with Hanks Balanced Salt Solution (HBSS) (GibcoBRL, Bethesda, Md.) and centrifuged at 40×g in 50 ml centrifuged tubes after each washing. 1-5 ml of pre-warmed trypsin-EDTA (Stemcell Technologies Inc, Vancouver, British Columbia, Canada) was added to the organoid pellet and was pipetted with P-1000 for 3 minutes, and then 10 ml of cold HBSS with 2% Fetal Bovine Serum (FBS) (Atlanta Biologicals, Norcross, Ga.) was added and centrifuged at 100×g for 5 min.

After centrifugation, the supernatant was removed, and 2-4 ml of pre-warmed dispase (Stemcell Technologies Inc, Vancouver, British Columbia, Canada) and 200-400 ul of 1 mg/ml DNAse 1 (Stemcell Technologies Inc, Vancouver, British Columbia, Canada) was added and pipetted for 1 minute. 10 ml of cold HBSS with 2% FBS was added and the cell suspension was filtered through a 40-μm cell strainer (Falcon) and then passed through a 22G pippetting needle with 90° blunt ends (Fisher Scientific) to obtain a single cell suspension.

An aliquot of the single cell suspension was mixed with trypan blue stain (GibcoBRL, Bethesda, Md.). In a hemocytometer, single cells, doublets, triplets, and groups of higher numbers of cells were counted. The number of single cells was >99% with >85% viability in all experiments. Single cells were plated in ultra-low attachment plates (Coming) or 0.6% agarose-coated plates at a density of 100,000 viable cells/ml in primary culture and 5000 cells/ml in subsequent passages.

For mammosphere culture, cells were grown in a serum-free mammary epithelial basal medium (MEBM) (Cambrex Bio Science Walkersville, Inc, Walkerville, Md.), supplemented with B27 (Invitrogen), 20 ng/mL EGF (BD Biosciences), antibiotic-antimycotic (100 unit/ml penicillin G sodium, 100 ug/ml streptomycin sulfate and 0.25 ug/ml amphotericin B) (GibcoBRL, Bethesda, Md.), 20 ug/ml Gentamycin, 1 ng/ml Hydrocortisone, 5 ug/ml Insulin and 100 μM 2-mercaptoethanol in a humidified incubator (10% CO2: 95% air, 37° C.). Mammospheres were collected by gentle centrifugation (1000 rpm) after 7-10 d and dissociated enzymatically (10 min in 0.05% trypsin, 0.53 mM EDTA; Invitrogen) and mechanically, using a pippetting needle with 90° blunt ends (Fisher Scientific). The cells obtained from dissociation were sieved through a 40-μm sieve and analyzed microscopically for single-cellularity. If groups of cells were present at a frequency >1%, mechanical dissociation and sieving were repeated. An aliquot of the cells was cultivated in suspension at a density of 5000 cells/ml. To induce cellular differentiation, 5×105 single cells were plated on a collagen-coated 60-mm plate and cells were cultured in Ham's F-12 medium (BioWhittaker) with 5% FBS, 5 μg/mL insulin, 1 μg/mL hydrocortisone, 10 ng/mL cholera toxin (Sigma), 10 ng/mL EGF (BD Biosciences), and 1× Pen/Strep/Fungizone Mix (BioWhittaker). After mammospheres were formed in suspension culture or cells reached 85% confluency on the collagen plate (about 7 d), total RNA was isolated using RNeasy Mini Kit (QIAGEN) and utilized for real-time quantitative RT-PCR assays.

Treatments of Mammospheres with Hedgehog and Notch Agonists and Antagonists

Single cells from epithelial organoids were plated in 6-well ultra-low attachment plates (Coming) at a density of 100,000 viable cells/ml. Cells were cultured in 2 ml of a serumfree MEBM per well. Biologically active, unmodified amino-terminal recombinant human Shh (Cat. 1314-SH, R&D Systems, Inc), recombinant mouse Ihh (Cat. 1705-HH R&D Systems, Inc), Cyclopamine (CP) from Toronto Research Chemicals Inc (Cat. C988400), the Notch peptide—Delta/Serrate/LAG-2 (DSL), and gamma secretase inhibitor (GSI) (Dontu et al., Breast Cancer Res. 2004;6(6):R605-15) were utilized. Cells were incubated for 7 days in the presence of different treatments as follows. For the treatment with Hedgehog agonists and antagonist, Shh was used at 1.5 μg/ml, 3 μg/ml and 6 μg/ml in the presence or absence of 300 riM of CP or 5 μM of GSI. CP was used at 150 nM, 300 nM and 600 nM concentrations, and the control was 12% 1×PBS with 0.06% BSA. For the treatment with Notch agonists and antagonist, DSL was used at 10 μM in the presence or absence of 5 μM of GSI or 300 nM of CP, and the controls consisted of 10 μM of scrambled Notch peptide. All treatments were continued for 10 days, with agonists and antagonists added every two or three days when medium was changed.

Mammospheres were then collected for in vitro self-renewal assays and Real-time quantitative RT-PCR. For reverse-transcriptase reactions, 1 μg of total RNA from mammospheres or differentiated cells on collagen-coated plates was reverse transcribed with 200 U M-MLV Reverse Transcriptase (GibcoBRL) at 42° C. for 1 hour in the presence of 5 mM each of dATP, dCTP, dGTP and dTTP, 4 μl 5× 1 st strand buffer (GibcoBRL), 0.01M DDT, 1 U RNA Guard RNase inhibitor (GibcoBRL), and 2.5 gM random primers in a total volume of 20 μl. The reaction was terminated by heating to 95° C. for 3 minutes. Real-time quantitative PCR (TaqMan™) primers and probes were purchased from AppliedBiosytems as Assays-on-Demand™ Gene Expression Products. Real-time PCRs were performed following the supplier's instructions (Applied Biosystems). 20 μl of PCR mixture contained 10 μl of 2× Taqman™ universal PCR Master Mix, 1 μl of 20× working stock of gene expression assay mix, and 50 ng of RNA converted cDNA. PCR was performed in a ABI PRISM® 7900HT sequence detection system with 384-Well block module and automation accessory (Applied Biosystems) by incubation at 50° C. for 2 min and then 95° C. for 10 min followed by 40 amplification cycles (15 s of denaturation at 95° C. and 1 min of hybridization and elongation at 60° C.). The reaction for each sample was performed in quadruplicates. Fluorescence of the PCR products was detected by the same apparatus. The number of cycles that it takes for amplification plot to reach the threshold limit, the Ct-value was used for quantification. RPLP0 was used for normalization.

Virus Production, Infection and Cell Culture

The retroviral plasmid DNAs for Vector only (SIN-IP-EGFP), Gli1 (SIN-GLI1-EGFP) (Regl et al., 2002, Oncogene, 21(36):5529-5539) and Gli2 (SIN-GLI2-EGFP) (Ikram et al., 2004, The Journal of Investigative Dermatology, 122(6):1503-1509) were generous gift from Dr. Graham W Neil. Retroviruses for SIN-IP-EGFP, SIN-GLI1-EGFP and SINGLI2-EGFP were produced by stable transfection in 293 cells and were utilized to infect the single cells isolated from primary mammosphere. Briefly, the plasmid DNAs were transfected into the 293 cells (Phoenix cells) by using the CalPhos™ Mammalian Transfection Kit from BD Biosciences Clontech and the transfected Phoenix cells were selected with 1.25 μg/ml puromycin 24 hours post-transfection. Viruses were collected when the cells were confluent. The collected viruses were concentrated by ultracentrifugation (20,000-30,000×g) for 3 hours, resuspended in serum-free MEBM and stored at −80° C. for the future use. On the day before virus transduction, primary mammospheres (about 7-10 days in suspension culture) were dissociated into single cells as described above, and the single cells were plated onto the 10-cm tissue culture coated plates at the density of 1 million cells/plate in Ham's F-12 medium (BioWhittaker) with 5% FBS, 5 μg/ml insulin, 1 μg/mL hydrocortisone, 10 μg/ml cholera toxin (Sigma), 10 ng/ml EGF (BD Biosciences), and 1× Pen/Strep/Fungizone Mix (BioWhittaker). After approximate 12-16 hours, the serum medium was removed and the cells were washed with 1×HBSS. The frozen concentrated retroviruses were quick thawed at 37° C. The cells were cultured in 6 ml of 1:1 ratio of retrovirus stock suspension culture MEBM in a humidified incubator (10% CO2: 95% air, 37(C). At the same time, Polybrene was added to a final concentration of 5 μg/ml. After 12-16 hour incubation, the cells were collected and resuspended in suspension culture MEBM at the density of 5000 cells/ml on 0.6% agarose-coated plates. After 7-10 days of cultivation, mammospheres were collected and used for the future assays immediately.

siRNA Contrustions

Three human Bmi-1 siRNA oligos were purchased from Ambion, Inc (Silencer™ Predesigned siRNAs, Ambion, Inc, Austin, Tex.) and were confirmed for the knock-down of Bmi-1 expression in human mammary epithelial cells from the reduction mammoplasties. The sequence of these three siRNA oligos is as follows: siRNA1-s: GGGTACTTCATTGATGCCA (SEQ ID NO:1); siRNA2-s: GGTCAGATAAAACTCTCCA (SEQ ID NO:2); and siRNA3-s: GGGCTTTTCAAAAATGAAA (SEQ ID NO:3). All of the siRNA sequences were converted to the small hairpin (shRNA) with the loop sequence of UUCAAGAGA and inserted as double-stranded DNA oligonucleotides into HpaI and XhoI sites of the lentivirus gene transfer vector LentiLox 3.7. All constructs were verified by sequencing. Because the green fluorescent protein (GFP) sequence is encoded in the lentivirus transduction vector under the control of a separate promoter, GFP is expressed in lentivirus-infected cells as the marker to indicate that the cells express the shRNA for human Bmi-1. Infected human mammary epithelia cells dissociated from reduction mammoplasties with these lentiviruses and performed the in vitro self-renewal assay as described above. In this Example, over 90% of cells were infected with the control (HIV-GFP-VSVG) or siRNA lentiviruses (HIV-siRNA1-VSVG, HIV-siRNA2-VSVG, HIV-siRNA3-VSVG).

3-D Matrigel Culture

3-D cultures in Matrigel were established as previously described (Weaver and Bissell, 1999, Journal of Mammary Gland Biology and Neoplasia, 4:193-201). Briefly, 30 mammospheres were suspended in 1 ml of BD Matrigel™ Matrix (Cat. 354234, BD Biosciences, Palo Alto, Calif.) and Ham's F-12 medium (BioWbittaker) with 5% serum at a ratio of 1:1, and plated 1 ml of the mixture into one well of 24-well cold plates and each group of mammospheres was performed in quadruplicates. After the matrigel was solidified, 1 ml of Ham's F-12 medium (BioWhittaker) with 5% serum was added to the top of the matrigel. The experiments were repeated with mammospheres derived from at least three different patients.

Mammosphere Implantation Into the Cleared Fatpads of NOD/SCID Mice

Three-week-old female NOD/SCID mice were anesthetized by an i.p. injection of ketamine/xylazine (30 mg ketamine combined with 2 mg of xylazine in 0.4-ml volume, which was diluted to 4 ml by using Hank's balanced salt solution (HBSS); 0.12 ml of the diluted solution was used per 12-g mouse), and the no. 4 inguinal mammary glands were removed from the mice. One 60-day release estrogen pellet (0.72 mg/pellet, Cat. # SE-121, Innovative Research of America, Sarasota, Fla.) was placed s.c on the back of the mouse's neck by using a trocar. At the same time, 400 mammospheres were mixed with 2.5×105 non-irradiated telomerase immortalized human mammary fibroblasts (a generous gift from John Stingl at Terry Fox Laboratory in Canada) and 2.5×105 irradiated (4 Gy) fibroblasts and resuspended in 10 μl of 1:1 matrigel (BD Biosciences, Palo, Alto, Calif.): Ham's F-12 medium (BioWhittaker) with 5% serum mixture and injected to each of the cleared fat-pad.

Whole Mounts, H&E Immunostaining

Approximate 8 weeks after the implantation, the fat-pad was removed and fixed in carnoy's solution for one hour at room temperature and subsequently stained with carmine alum overnight. The tissue was then defatted through graded ethanol and cleared in 5 ml of xylene for one hour, and the whole mount pictures were taken with an Olympus BX-51 microscope. The tissue was then embedded in the paraffin and sectioned for H&E staining.

Preparation of Single Cell Suspensions of Tumor Cells, Xenografts and Flow Cytometry

All animal studies were carried out under the approved institutional animal protocols and the mice were prepared for the xenografts as described by Al-Hajj (Al-Hajj et al., 2003, PNAS USA, 100(7), 3983-3988). The original tumor cells from the xenograft tumors were a generous gift from Dr. Michael Clarke's laboratory at University of Michigan and we passaged these tumor cells several times in NOD/SCID mice as described previously (Al-Hajj et al., 2003). Following tumor growth, which took 1-2 months, tumors were removed. Before digestion with collagenase, xenograft tumors were cut up into small pieces and then minced completely by using sterile blades. To obtain single cell suspensions, the resulting tumor pieces were then transferred to a small tissue dissociation flask with collagenase type 3 (Worthington Biochemical Corporation, Lakewood, N.J.) in medium DMEM/F12 (300 units of collagenase per ml) and allowed to incubate at 37° C. for 3-4 h on a rotary shaker. Every one hour, pipetting with a 10-ml pipette was done, and cells were filtered through a 40-μm sieve and stored in RPMI/20% FBS at 4° C. At the end of the incubation, all of the sieved cells were washed with RPMI/20% FBS, then washed twice with HBSS. One part of cells were used for flow cytometry to sort out the H2Kd-CD44+CD24−/lowLineage−population and H2Kd-CD44-/lowCD24+Lineage+population as described previously (Al-Hajj et al., 2003), and the RNA were extracted from these two populations and real-time RT-PCR were used to determine the gene expression; one part of cells were used for flow cytometry to sort out PTCH1+Ihh+population and PTCH1−Ihh−population, and the sorted two populations were separately injected to each side of the mouse fat pads as described previously (Al-Hajj et al., 2003); and the rest of the cells were frozen for the future use. Once the biggest tumors reached to about 8-mm diameter, the tumors were removed and single cell suspensions were prepared from each group of tumors and used for flow-cytometry analysis as described above.

Statistical Analysis

Results are presented as the mean +standard deviation (STEV) for at least 3 repeated individual experiments for each group. Analysis was performed using Minitab statistical software for Windows (Minitab Inc., State College, Pa.). Statistical differences were determined by using one-way ANOVA for independent samples. p-values and &-values of less than 0.05 were considered statistically significant.

Results

Hedgehog Pathway Genes are Highly Expressed in Mammary Stem/Progenitor Cells

In order to compare expression of genes in the Hedgehog pathway in mammary stem/progenitor cells and differentiated mammary cells, primary mammospheres were disassociated and part of the single cells were cultured in suspension on non-adherent plates in serum-free MEBM as secondary mammospheres (mammary stem/progenitor cells), and part of the single cells were cultured in suspension on a collagen substratum in serum containing medium (differentiated mammary cells). It has been previously demonstrated that secondary mammospheres are composed of stem and progenitor cells as demonstrated by the ability of these cells to undergo self-renewal and multilineage differentiation (Dontu et al., 2003, Genes and Development, 17(10), 1253-1270). In contrast, attachment of cells to collagen substrata induces irreversible differentiation of these cells (Dontu et al., 2003).

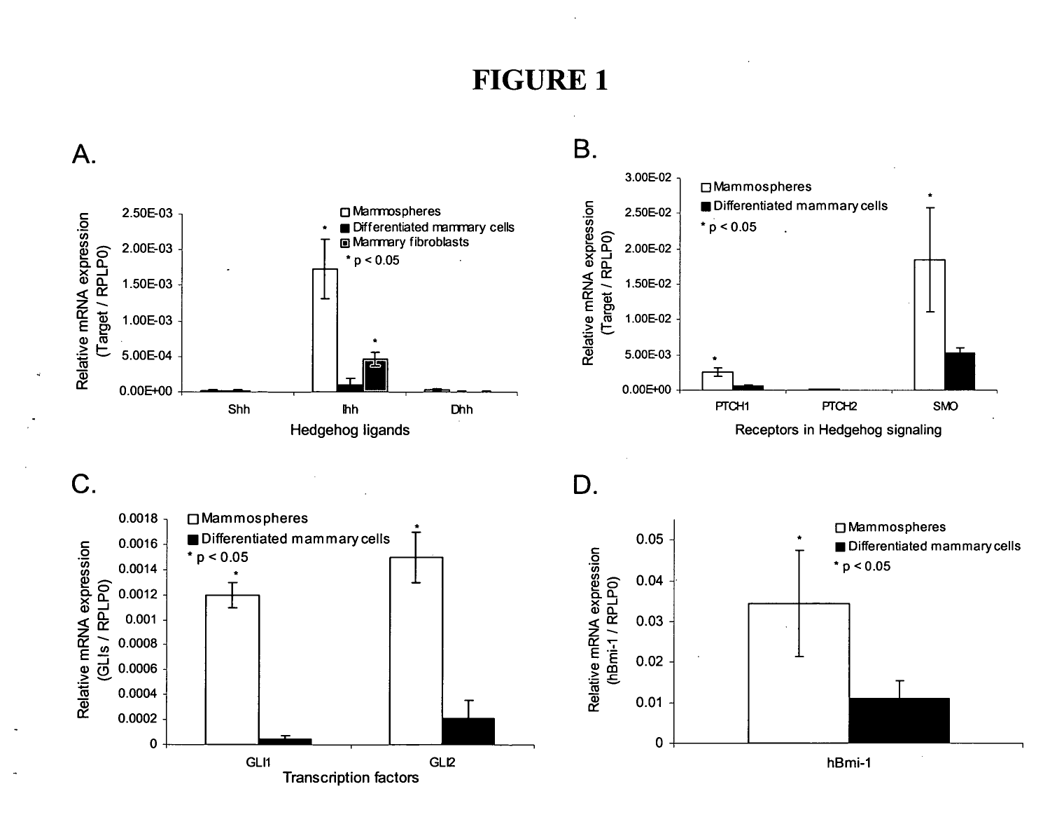

mRNA levels were determined by real-time quantitative RT-PCR in mammary stem/progenitor cells and differentiated mammary cells isolated from reduction mammoplasty tissues. As shown in FIG. 1A, Ihh (Indian Hedghog) is the major ligand expressed and is expressed at approximate 9 fold higher level in stem/progenitor cells in mammospheres compared to differentiated cells cultured on a collagen substrate. Interestingly, Ihh is also expressed in mammary fibroblasts although at lower level than in mammospheres. This indicates that there may be paracrine Hedgehog signaling between mammary epithelial cells and fibroblasts, as well as signaling between the epithelial components of mammospheres. FFIG. 1B shows that hedgehog receptors PTCH1, PTCH2 and SMO are expressed in both cell populations; however, mammary stem/progenitor cells in mammospheres express about 4-fold higher levels of PTCH1 and PTCH2 mRNA, and 3-fold higher levels of SMO mRNA compared to differentiated mammary cells on collagen substrata. The mRNA expression of hedgehog downstream transcription factors Gli1 and Gli2 was measured, demonstrating that mammary stem/progenitor cells have almost 25-fold higher levels of Gli1 mRNA and 6-fold higher levels of Gli2 mRNA than differentiated mammary cells (FIG. 1C). This indicates that the Hedgehog signaling pathway is activated in the mammary stem/progenitor cells compared to the differentiated mammary cells, indicating that the hedgehog pathway might regulate mammary stem cell self-renewal. In hematopoitic and neural stem cells, the polycomb gene Bmi-1 has been shown to be required for stem cell self-renewal. Interestingly, it was found that Bmi-1 mRNA levels are increased about 3.5 fold in mammary stem/progenitor cells (FIG. 1D), which indicates that Bmi-1 may be a downstream target of the hedgehog pathway in the regulation of stem cell self-renewal.

Hedgehog Signaling Agonists and Antagonist Regulate Self-Renewal of Mammary Stem Cells

The mammosphere-based culture system were utilized to examine the role of Hedgehog signaling in mammary stem cell self-renewal. It has been previously shown that mammospheres could be passaged at clonal density and at each passage new mammospheres were generated, consisting of cells with multilineage differentiation potential (Dontu et al., 2003, Genes and Development, 17(10), 1253-1270) and Dontu et al., 2004, Breast Cancer Research, 6(6):R605). These studies suggested that mammospheres are composed of a small number of stem cells with the remainder consisting of progenitors capable of multilineage differentiation but not sphere formation. It has been previously shown that mammosphere number reflects stem cell self-renewal, whereas mammosphere size reflects progenitor proliferation (Dontu et al., 2003 and Dontu et al., 2004). The dose effects of the hedgehog ligand—Shh (Sonic Hedgehog) and Hedgehog signaling inhibitor—Cyclopamine (CP) were examined on primary and secondary mammosphere formation. Primary mammospheres were formed in the presence of the Shh, Cyclopamine or both. These mammospheres were then dissociated into single cells and the number of secondary mammospheres produced was determined.

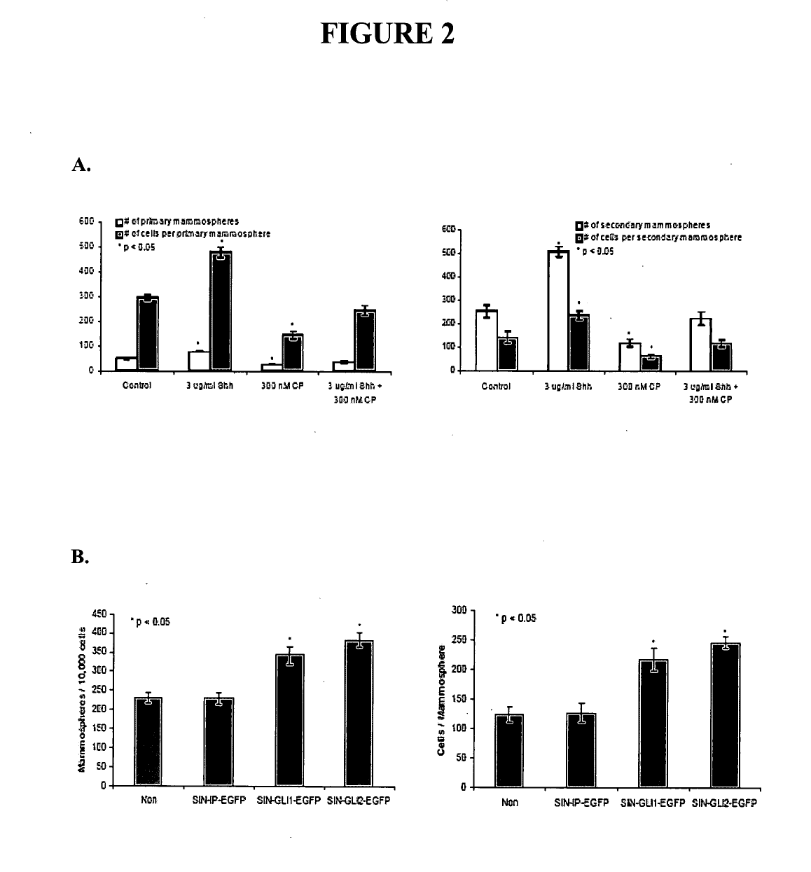

Different concentrations of Shh (1.5 μg/ml, 3 μg/ml, 6 μg/ml) and Cyclopamine (150 nM, 300 nM, 600 nM) were tested and it was found that both 1.5 μg/ml of Shh and 150 nM of Cyclopamine had no effects on the mammosphere formation and the other two doses had significant effects. Therefore, 3 μg/ml of Shh and 300 nM of Cyclopamine were utilized. We found that;, in comparison to the control, Shh increased primary mammosphere formation by 57% and increased the average cell number in these mammospheres by 62% (FIG. 2A). In contrast, the Hh pathway inhibitor, Cyclopamine, decreased primary mammosphere formation by 45% and decreased the average cell number in the primary mammospheres by 51% (FIG. 2A). The specificity of Cyclopmaine inhibition was demonstrated by the reversal of inhibition by the addition of 3 μg/ml of Shh (FIG. 2A).

To more directly demonstrate a role for the Hedgehog signaling in the regulation of mammary stem cell self-renewal in vitro, the effect of pathway activation or inhibition on secondary mammosphere formation was determined. It was previously demonstrated that the ability to clonally generate mutilineage mammospheres that can be serially passaged is a measure of the self-renewal capacity of the mammosphere initiating cells (Dontu et al., 2003). It was determined that in comparison to the control group, single cells from the Shh-treated primary mammospheres formed 100% more secondary mammospheres and the average cell numbers per secondary mammosphere were increased 67% (FIG. 2A). In contrast, single cells from primary mammospheres treated with Cyclopamine generated 54% less secondary mammospheres and the average cell numbers per secondary mammosphere were decreased 56% (FIG. 2A) compared to controls. This inhibition could be reversed by addition of 3 μg/ml of Shh (FIG. 2A). The ability of Hedgehog ligand Shh and Hedgehog inhibitor Cyclopamine to regulate mammosphere formation indicates that Hedgehog activation promotes mammary stem cell self-renewal. Since Ihh was the most abundant Hedgehog ligand expressed in the mammospheres as assayed by real-time quantitative RT-PCR, we also determined the effect of recombinant Ihh on the system. The effects of Ihh on mammosphere formation were similar to those of Shh.

Mammary Stem Cell Self-Renewal is Regulated by Gli Transcription Factors

As indicated above, activation of Hh signaling increased expression of the downstream transcription factors Gli1 and Gli2 and stimulated mammary stem cell self-renewal. In order to determine whether the increase in stem cell self-renewal was mediated by these transcription factors, mammosphere were infected by initiating cells with retro-viral vectors containing Gli1 or Gli2 and determined the effect of constitutive expression of these transcription factors on mammosphere formation.

A highly efficient retroviral expression system was used to generate Gli1-expressing, Gli2-expressing and EGFP (enhanced GFP)-expressing human mammospheres. It was found that in comparison to the uninfected controls or the EGFP-expressing group, overexpression of Gli1 and Gli2 stimulated mammosphere formation by 49% and 66% respectively (FIG. 2B). Furthermore, overexpression of Gli1 and Gli2 increased the mammosphere size by 77% and 100% respectively (FIG. 2B). These results indicate that the Hedgehog regulation of mammary stem cell self-renewal and progenitor proliferation are mediated by the downstream transcription factors Gli1 and Gli2.

Hedgehog Signaling Promotes Branching Morphogenesis

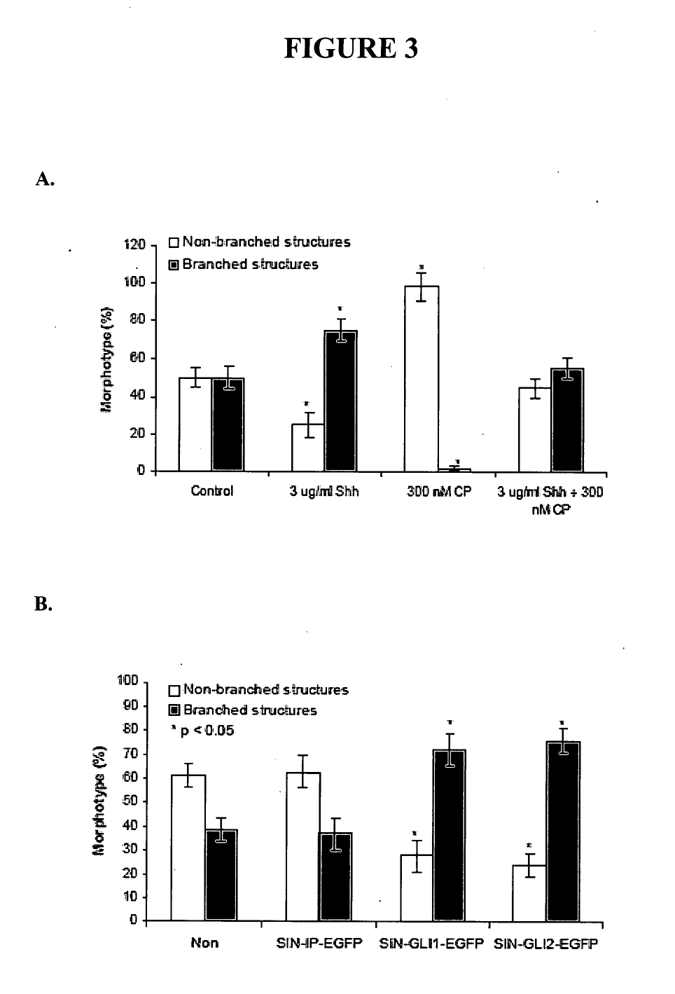

Reconstituted basement membrane (Matrigel) has been demonstrated to promote morphogenic differentiation of human or rodent mammary cells (Gudjonsson et al., 2002, Genes and Development, 16, 693-706). Following three weeks of cultivation in Matrigel, some mammospheres developed extensive ductal lobulo-alveolar structures similar in morphology to structures found in vivo, whereas, others produced hollow alveolar structures that fail to branch. This system was utilized to examine the role of the Hedgehog signaling in branching morphogenesis. It was determined that the activation of the Hedgehog signaling by either the addition of Shh or the overexpression of Gli1 or Gli2 facilitated branching morphogenesis in this system. Addition of Shh increased branching by 50% (FIG. 3A) and overexpression of Gli1 or Gli2 increased branching by 100% (FIG. 3B). In addition to increasing the number of branched structures, activation of Hh signaling increased the length of these structures (FIG. 3). Interestingly, Cyclopamine almost completely blocked branch formation. While not limited to any mechanism, and not necessary to practice the present invention, it is believed that these results indicate that Hh signaling is important for branching morphogenesis in this system.

Gli-Overexpression in Mammary Stem Cells Promotes Ductal Hyperplasia in Humanized NOD-SCID Mouse Mammary Fat Pads

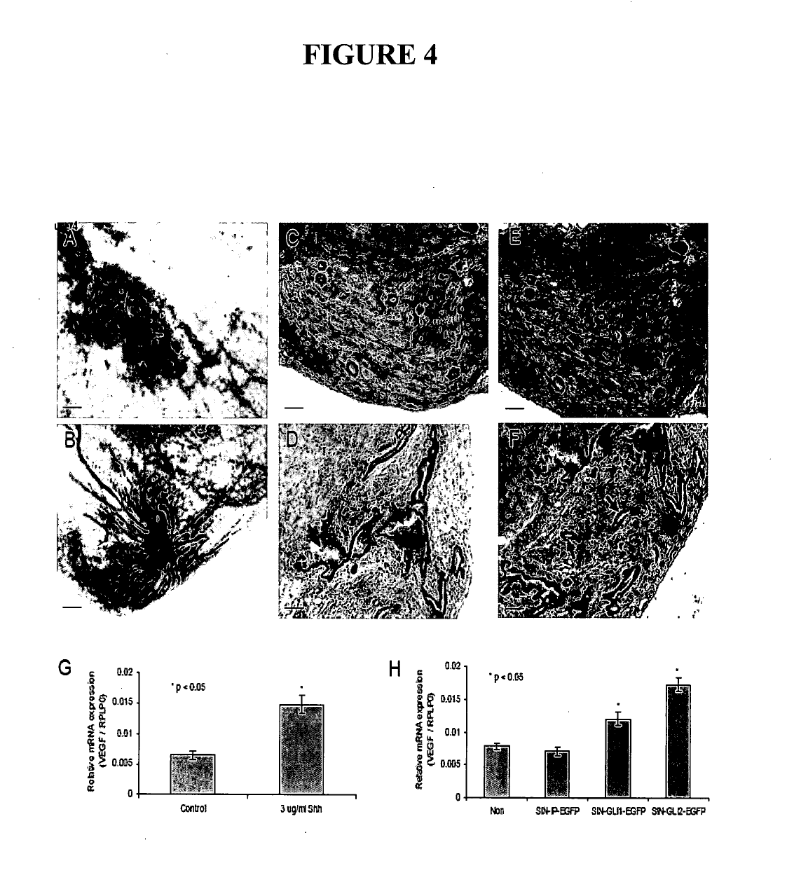

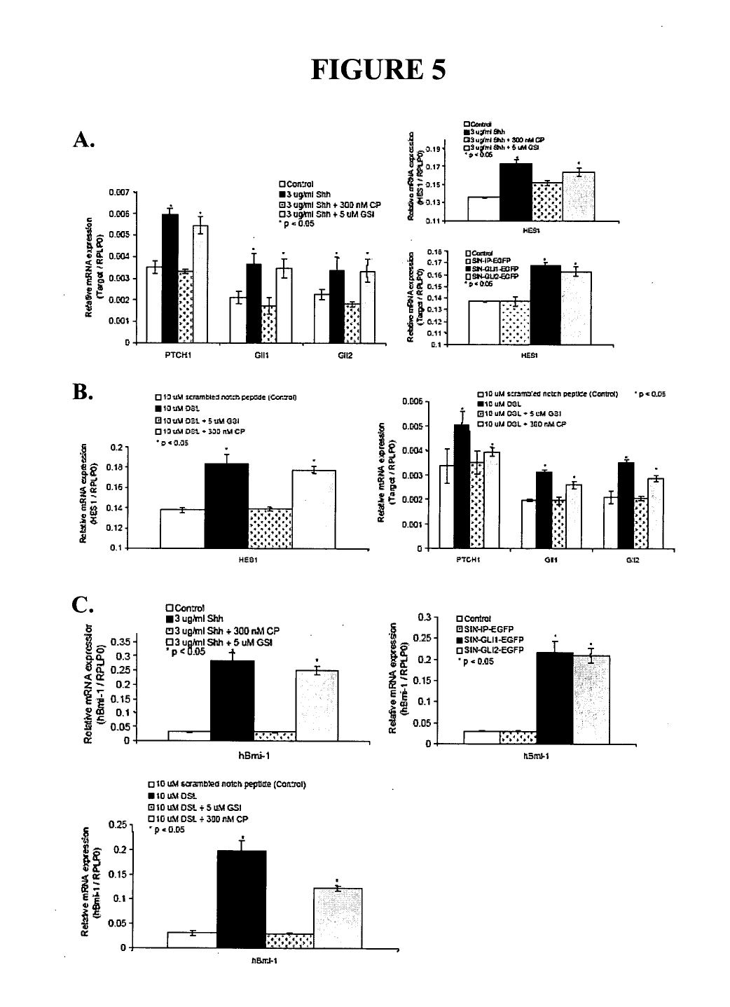

In order to determine the effects of Gli-overexpression on mammary development, a system has been developed in which mammospheres can be implanted into the humanized fat pads of NOD-SCID mice. This system is a modification of that described recently by Kuperuasser in which human mammary fibroblasts are implanted into the cleared fat pads of NOD-SCID mice were able to support the growth of human mammary epithelial cells (Kuperwasser et al., 2004, PNAS, USA, 101(14), 4966-4971). The cleared fat pads of three-week old NOD-SCID mice were humanized with telomerase immortalized human mammary fibroblasts. At the same time, control mammospheres or those overexpressing Gli1 or Gli2 were introduced into these humanized fat pads of mice implanted with an estrogen pellet. After eight weeks, the mammary glands were removed and examined by whole mount and histologic analysis. The histology of these explants was compared to normal mouse and human mammary glands. In the normal mouse mammary gland, mouse epithelial structures are surrounded by a sparse mouse stroma which is considerably less dense than human stroma which surrounds human epithelial structures. Dense human mammary stroma was apparent in the humanized NOD-SCID mouse fat pad (FIGS. 4C, 4D, 4E, 4F). Control mammospheres (SIN-IP-EGFP) produced limited ductal growth in areas surrounded by dense human mammary stroma (FIG. 4A and 4C). In contrast, Gli2-overxpressing mammospheres (SIN-GLI2-EGFP) developed substaintually more branching structures (FIGS. 4B and 4D) than control mammospheres. Microscopic examination indicated that Gli2 transfected mammospheres produced ductal hyperplasia. In addition, there was an increased density of blood vessels in the stroma surrounding hyperplastic structures in the Gli2 transfected mammospheres (FIG. 4F) compared to the control (FIG. 4E). In the in vivo studies, we found Gli1 has less effects on human mammary outgrowths and blood vessel formation compared to Gli2. These studies demonstrate that mammospheres can generate human ductal alveolar structures when implanted into the humanized cleared fat pad of NOD-SCID mice. Furthermore, overexpression of Gli2 in mammospheres is sufficient to induce ductal hyperplasia in these outgrowths.

Hedgehog Activation Promotes VEGF Production and Angiogenesis

As indicated above it was noted that in addition to producing ductal hyperplasias mammopsheres transfected with Gli2 displayed increased blood vessel density in the stroma-surrounding human xenografts. In order to determine the mechanism of this angiogenic response, the effect of hedgehog activation on VEGF production by mammospheres in vitro was examined. Addition of recombinant Shh increased VEGF mRNA levels by almost three-fold (FIG. 4G). VEGF mRNA was also increased in Gli1 and Gli2-overexpressing mammospheres compared to controls (FIG. 4H). This indicates that the increased vascular structures seen in Gli2 transfected xenografts may be accounted for by Hh induction of VEGF.

Hedgehog and Notch Signaling Pathways Demonstrate Bi-Directional Interaction

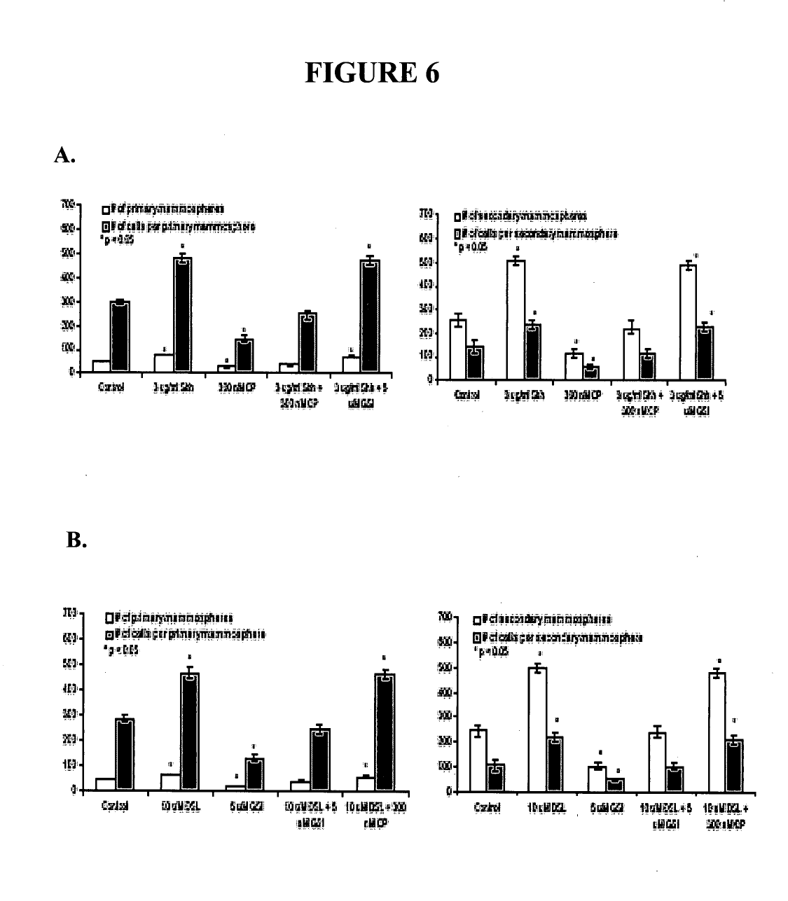

It has previously been shown that Notch signaling could act on mammary stem cells to promote their self-renewal (Dontu et al., 2004). Since Hedgehog signaling also appears to regulate this process, it was determined whether there are interactions between Hedgehog and Notch signaling pathways. In order to demonstrate interaction between these pathways, a Notch agonist (DSL) was utilized (Dontu et al., 2004) in the absence or presence a Notch antagonist (GSI) (Dontu et al., 2004) or a Hedgehog antagonist (Cyclopamine) to determine their effects on mammary stem cell self-renewal as well as on the expression of genes involved in the Hh and Notch signaling. A Hh agonist (sonic Hedgehog) was also utilized in the absence or presence a Hedgehog antagonist (Cyclopamine) or a Notch antagonist (GSI) (Dontu et al., 2004) to determine their effects on mammary stem cell self-renewal and genes involved in the Notch and Hh signaling pathway. It was found that activation of Hedgehog signaling by the addition of Shh increased mRNA expression of Hh pathway components PTCH 1, Gli1, and Gli2 (FIG. 5A). In addition to activating Hh genes, the addition of Shh also significantly increased the expression of the Notch downstream target HES1 (FIG. 5A). All of these affects were partially blocked by the Hh inhibitor Cyclopamine, but not by the Notch pathway inhibitor GSI (FIG. 5A). The Notch target HES1 was also increased in Gli1- and Gli2-overexpressing mammospheres (FIG. 5A). In order to determine whether activation of the Notch pathway could affect Hh targets, Notch signaling was activated by utilizing the DSL ligand which binds to all Notch receptors (Dontu et al., 2004). Activation of Notch by DSL increased the expression of the Notch downstream transcription factor HES1 (FIG. 5B), but also increased expression of mRNA for the Hh pathway targets PTCH1, Gli1 and Gli2 (FIG. 5B). This activation could be completely blocked by the Notch pathway inhibitor GSI and partially blocked by the Hh signaling inhibitor Cyclopamine (FIG. 5B). These results indicate that the Notch and Hh pathways are able to interact in a bi-directional manner. As shown in FIG. 6A, the Notch inhibitor GSI did not block the effects of the Hedgehog ligand Shh on mammary stem cell self-renewal. It was then determined whether the Hedgehog inhibitor Cyclopamine could have effects on the activation of the Notch signaling by the Notch Ligand DSL. In FIG. 6B, previous findings were confirmed that DSL could stimulate mammary stem cell self-renewal (Dontu et al., 2004), which can be blocked by GSI, but not by Cyclopamine.

The Polycomb Gene Bmi-1 is Downstream of Hh and Notch Signaling

Bmi-1 is a polycomb gene, which functions as a transcriptional repressor. Recently, it has been shown that Bmi-1 regulates and is required for self-renewal of hematopoitic (Park et al., 2003, Nature, 423, 302-305) and neural stem cells (Molofsky et al., 2003, Nature, 425 (6961):9620967). Furthermore, it has recently been shown that Bmi-1 expression is increased upon the addition of Sonic Hedgehog or on overexpression of the Sonic Hedgehog target Gli in cerebellar granular cells (Leung et al., 2004, Nature, 428:337-341). Therefore, the effect of Hedgehog activation on Bmi-1 expression was assayed. It was determined that activation of the Hedgehog pathway by addition of Shh resulted in a 8-fold increase in expression of Bmi-1 in mammospheres, an effect that was blocked by the Hedgehog pathway specific inhibitor Cyclopamine, but not by the Notch pathway specific inhibitor GSI (FIG. 5C). Furthermore, both Gli1 overexpressing and Gli2-overexpressing mammospheres displayed a 6-fold higher Bmi-1 expression compared to control cultures (FIG. 5C). Together, these studies demonstrate that Bmi-1 expression can be regulated by Hh signaling.

As indicated above, it was found that there are interactions between the Notch and Hh pathways. The effect of Notch activation on Bmi-1 expression was therefore determined. Activation of the Notch pathway by DSL increased Bmi-1 expression by 5-fold, an effect which could be completely blocked by GSI, but not by Cyclopamine (FIG. 5C). Taken together, these studies provide further evidence for bi-directional interactions between the Hh and Notch pathways with subsequent regulation of the downstream target Bmi-1.

Effects of Hh and Notch Signaling on Mammary Stem Cell Self-Renewal are Mediated by Bmi-1

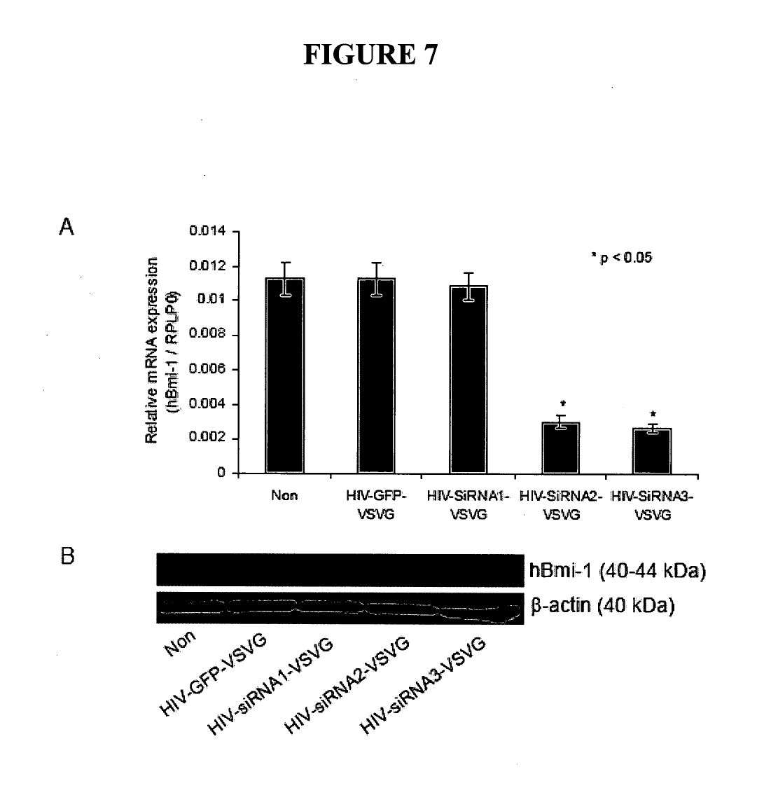

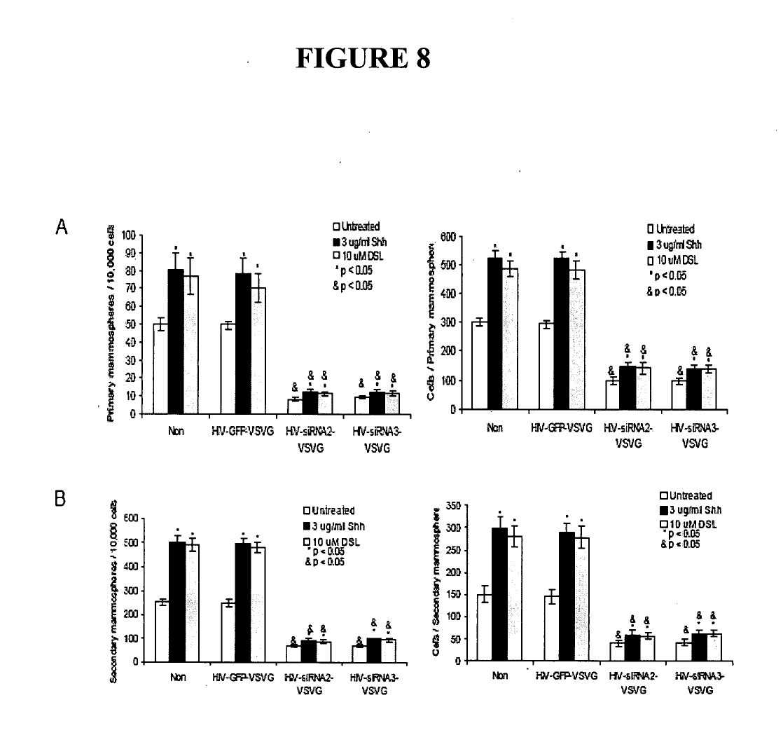

In order to show that Hh and Notch pathway effects on stem cell self renewal are mediated by Bmi-1, siRNA was utilized that was delivered in a lentiviral vector tagged with a GFP to down regulate Bmi-1 expression in mammospheres. This vector has over 90% transfection efficiency as determined by GFP expression. Both realtime PCR and western blotting were utilized to confirm the Bmi-1 knock-down by these siRNA lentiviruses in the mammosphere system, and two different siRNA lentiviruses significantly reduced the Bmi-1 expression at both mRNA level (over 80% reduction) and protein level (over 70% reduction) (see FIG. 7). These vectors were utilized to examine the effect of down regulation of Bmi-1 on mammosphere formation in the presence or absence of Hh or Notch activation. Down regulation of Bmi-1 expression reduced primary and secondary mammosphere formation by 80% (FIG. 8A) and 70% (FIG. 8B), respecitively; and reduced the primary and secondary mammosphere size by 60% (FIG. 6A) and 70% (FIG. 8B), respectively (FIG. 8B). Furthermore, the effects of Hh and Notch activation on both primary and secondary mammosphere formation was significantly reduced by Bmi-1 down regulation (FIG. 8). These experiments indicate that Hh and Notch mediate stem cell self-renewal through regulation of polycomb gene Bmi-1.

The Hedgehog Pathway and Bmi-1 are Activated in Breast Tumor Stem Cells

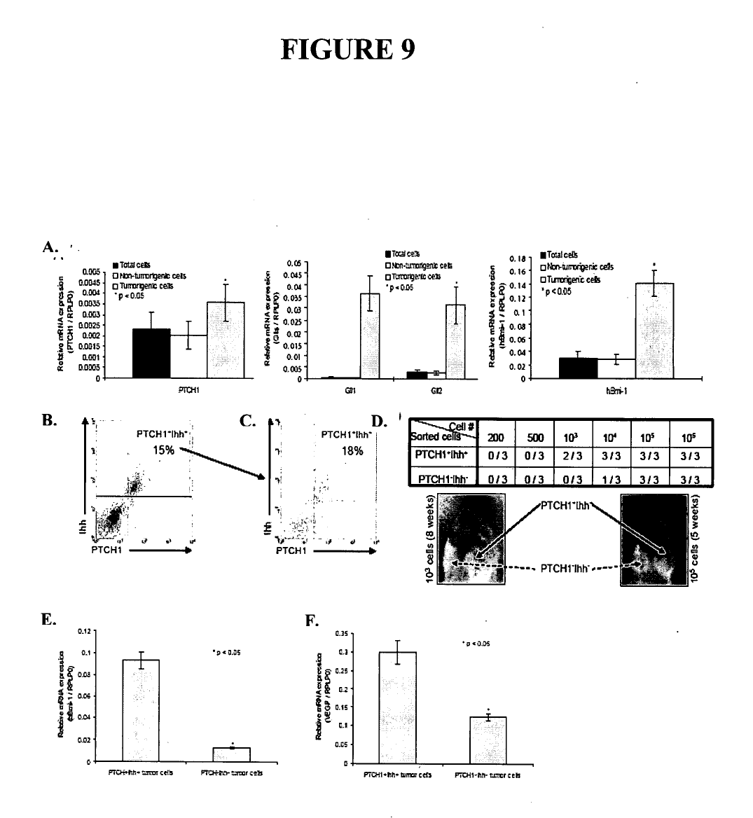

It has recently been reported that human breast cancers are driven by a small subset of “tumor stem cells” which are characterized by the cell surface phenotype CD44+CD24−/lowlin-. These cells functionally resemble normal stem cells in that they are able to selfrenew as well as to differentiate into non-tumorigenic cells which form the bulk of tumors (Al-Hajj et al., 2003). In order to determine whether the Hh pathway is activated in tumor stem cells, flow cytometry was utilized to isolate tumor stem cells expressing these cell surface markers from a human tumor xenograft derived from a metastatic human breast carcinoma utilizing these cell surface markers. mRNAs for Hh pathway components and Bmi-1 were measured by real-time PCR. As indicated in FIG. 9A, “tumor stem cells” displayed increased expression of Hh pathway components PTCH1 and Gli1 and an 8-fold increase in Bmi-1 compared to the cells isolated from the same tumor, which lacked the tumor stem cell markers (FIG. 9A).

In order to provide more evidence that tumor cells with activated Hh pathway components displayed “tumor stem cell properties”, flow cytometry was utilized to isolate tumor cells that displayed Hh activation. As shown in FIG. 9B, approximately 15% of cells from tumor xenografts displayed increased expression of the ligand lhh as well as the Hh receptor PTCH1. In order to determine whether these cells had increased tumoregenic capacity, PTCH1+Ihh+cells were isolated by flow cytometry and serial dilutions of these cells were injected into the fat pads of NOD-SCID mice. The same number of PTCH1−Ihh−cells were injected into the contralateral mammary fat pads. As noted in FIG. 9D, PTCH1+Ihh+cells gave rise to significantly more and larger tumors compared to PTCH1−Ihh−cells derived from the same tumor.

In addition to demonstrating self-renewal as indicated by ability to be serially transplanted in NOD-SCID mice, a predicted property of “tumor stem cells” is their ability to differentiate into the nontumoregenic cells which form the bulk of the tumor (Al-Hajj et al., 2003). In order to access this, tumors derived from PTCH1+Ihh+cells were isolated and their expression of Hh components evaluated by flow cytometry. As indicated in FIG. 9C, these tumors displayed expression patterns of PTCH+Ihh+ as well as PTCH1−Ihh− which resembled those of the initial tumor. Furthermore, as previously seen in CD44+CD24−/lowLin-tumor cells, PTCH1+Ihh+tumor cells displayed increased expression of Bmi-1 (about 9-fold increase) compared PTCH1-Ihh-cells from the same tumor (FIG. 9E). These studies indicate that tumor cells with activated Hh signaling components behave as “tumor stem cells” that are able to self-renew as well as to differentiate into cells that constitute the bulk of the tumor.

PTCH1+Ihh+Tumor Stem Cells Expressed Increased Levels of VEGF

As described above, activation of Hh signaling in normal breast stem/progenitor cells in mammospheres results in increased production of VEGF, a potent angiogenesis factor. As was found for normal human mammary stem cells, PTCH1+Ihh+“tumor stem cells” expressed 250% more VEGF mRNA than did PTCH1−Ihh−tumor cells (FIG. 9F). While not limited to any mechanism, and not necessary to practice the present invention, it is believed that these studies indicate that the activation of Hh signaling components in tumor stem cells plays a role in tumor angiogenesis in addition to facilitating tumor stem cell self-renewal.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

We claim:

1. A method of reducing or eliminating tumorigenic cells in a subject, comprising: administering a hedgehog signaling pathway antagonist to said subject under conditions such that at least a portion of said tumorigenic cells are killed, inhibited from proliferating, or from causing metastasis.

2. The method of claim 1, wherein said tumorigenic cells are mammary progenitor cells.

3. The method of claim 1, wherein said hedgehog signaling pathway antagonist comprises an antibody or antibody fragment.

4. The method of claim 1, wherein said hedgehog signaling pathway antagonist comprises Cyclopamine or a Cyclopamine antagonist.

5. The method of claim 1, wherein said tumorigenic cells are mammary cells characterized by an increased level of expression of a hedgehog signaling pathway component compared to non-tumorigenic mammary cells from said subject.

6. The method of claim 1, wherein said hedgehog signaling pathway component is selected from the group consisting of: PTCH1, Ihh, Gli1, Gli1, Bmi-1, and VEGF.

7. The method of claim 1, further comprising surgically removing a tumor from said subject prior to said administering.

8. A method for screening a compound, comprising: a) exposing a sample comprising a tumorigenic mammary cell to a candidate anti-neoplastic compound, wherein said candidate anti-neoplastic compound comprises a hedgehog signaling pathway antagonist; and b) detecting a change in said cell in response to said compound.

9. The method of claim 8, wherein said sample comprises a non-adherent mammosphere.

10. The method of claim 8, wherein said hedgehog signaling pathway antagonist comprises an antibody or antibody fragment.

11. The method of claim 8, wherein said hedgehog signaling pathway antagonist comprises a Cyclopamine analog.

12. The method of claim 8, wherein said sample comprises human breast tissue.

13. The method of claim 8, wherein said detecting comprises detecting cell death of said tumorigenic breast cell.

14. The method of claim 13, further comprising identifying said candidate anti-neoplastic agent as capable of killing tumorigenic cells.

15. A method of obtaining an enriched population of progenitor cells, comprising a) providing an initial sample comprising progenitor and non-progenitor cells, and b) sorting said initial sample based on the expression level of a hedgehog signaling pathway component expression in said cells such that an enriched population is generated, wherein said enriched population contains a higher percentage of progenitor cells than present in said initial sample.

16. The method of claim 15, wherein said sorting comprises the use of flow cytometry.

17. The method of claim 15, wherein said sorting comprises the use of immuno-magnetic sorting.

18. The method of claim 15, wherein said progenitor cells comprise tumorigenic cells and said non-progenitor cells comprise non-tumorigenic cells.

19. The method of claim 15, said hedgehog signaling pathway component is selected from the group consisting of: PTCH1, Ihh, Gli1, Gli1, Bmi-1, and VEGF.
